(19) United States
(12) Patent Application Publication
(10) Pub. No.: US 2013/0273052 A1
(43) Pub. Date: Oct. 17, 2013

(54) POLYPEPTIDES THAT BIND TO HUMAN COMPLEMENT COMPONENT C5

(71) Applicants: David Gies, Southbury, CT (US); Jeffrey W. Hunter, New Britain, CT (US); Jeremy P. Springhorn, Guilford, CT (US)

(72) Inventors: David Gies, Southbury, CT (US); Jeffrey W. Hunter, New Britain, CT (US); Jeremy P. Springhorn, Guilford, CT (US)

(73) Assignee: ALEXION PHARMACEUTICALS, INC., Cheshire, CT (US)

(21) Appl. No.: 13/877,298

(22) PCT Filed: Sep. 30, 2011
(86) PCT No.: PCT/US11/54143

§ 371 Date: Jun. 5, 2013

Related U.S. Application Data
(60) Provisional application No. 61/388,902, filed on Oct. 1, 2010.

(51) Int. Cl. C07K 16/38; A61K 45/06; A61K 39/395

(52) U.S. Cl. CPC C07K 16/38; A61K 39/3955; A61K 45/06
USPC 424/135.1; 530/387.3; 536/23.53; 435/320.1; 435/69.6; 435/375; 530/391.1; 435/254.21; 435/252.33; 435/254.23; 435/328

(57) ABSTRACT

The present disclosure relates to, inter alia, C5-binding polypeptides and use of the polypeptides in methods for treating or preventing complement-associated disorders. Also featured are therapeutics kits containing one or more of the C5-binding polypeptides and means for administering the polypeptides to a subject.

### CROSS-REFERENCE TO RELATED APPLICATIONS

This application claims priority to and the benefit of U.S. provisional patent application Ser. No. 61/388,902 filed on Oct. 1, 2010, the disclosure of which is incorporated herein by reference in its entirety.

### TECHNICAL FIELD

The field of the invention is medicine, immunology, molecular biology, and protein chemistry.

### BACKGROUND

The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory, and lytic functions. A concise summary of the biologic activities associated with complement activation is provided, for example, in The Merck Manual, 16^{th} Edition.

The complement cascade can progress via the classical pathway (CP), the lectin pathway, or the alternative pathway (AP). The lectin pathway is typically initiated with binding of mannose-binding lectin (MBL) to high mannose substrates. The AP can be antibody independent, and can be initiated by certain molecules on pathogen surfaces. The CP is typically initiated by antibody recognition of, and binding to, an antigenic site on a target cell. These pathways converge at the C3 convertase—the point where complement component C3 is cleaved by an active protease to yield C3a and C3b.

The AP C3 convertase is initiated by the spontaneous hydrolysis of complement component C3, which is abundant in the plasma in the blood. This process, also known as “tickover,” occurs through the spontaneous cleavage of a thioester bond in C3 to form C3i or C3(H₂O). Tickover is facilitated by the presence of surfaces that support the binding of activated C3 and/or have neutral or positive charge characteristics (e.g., bacterial cell surfaces). This formation of C3(H₂O) allows for the binding of plasma protein Factor B, which in turn allows Factor D to cleave Factor B into Ba and Bb. The Bb fragment remains bound to C3 to form a complex containing C3(H₂O)Bb—the “fluid-phase” or “initiation” C3 convertase. Although only produced in small amounts, the fluid-phase C3 convertase can cleave multiple C3 proteins into C3a and C3b and results in the generation of C3b and its subsequent covalent binding to a surface (e.g., a bacterial surface). Factor B bound to the surface-bound C3b is cleaved by Factor D to thus form the surface-bound AP C3 convertase complex containing C3b,Bb. (See, e.g., Müller-Eberhard (1988) Ann Rev Biochem 57:321-347.)

The AP C5 convertase—(C3b)₂,Bb—is formed upon addition of a second C3b monomer to the AP C3 convertase. (See, e.g., Medicus et al. (1976) J Exp Med 144:1076-1093 and Fearon et al. (1975) J Exp Med 142:856-863.) The role of the second C3b molecule is to bind C5 and present it for cleavage by Bb. (See, e.g., Isenman et al. (1980) J Immunol 124:326-331.) The AP C3 and C5 convertases are stabilized by the addition of the trimeric protein properdin as described in, e.g., Medicus et al. (1976), supra. However, properdin binding is not required to form a functioning alternative pathway C3 or C5 convertase. See, e.g., Schreiber et al. (1978) Proc Natl Acad Sci USA 75: 3948-3952 and Sissons et al. (1980) Proc Natl Acad Sci USA 77: 559-562.

The CP C3 convertase is formed upon interaction of complement component C1, which is a complex of C1q, C1r, and C1s, with an antibody that is bound to a target antigen (e.g., a microbial antigen). The binding of the C1q portion of C1 to the antibody-antigen complex causes a conformational change in C1 that activates C1r. Active C1r then cleaves the C1-associated C1s to thereby generate an active serine protease. Active C1s cleaves complement component C4 into C4b and C4a. Like C3b, the newly generated C4b fragment contains a highly reactive thiol that readily forms amide or ester bonds with suitable molecules on a target surface (e.g., a microbial cell surface). C1s also cleaves complement component C2 into C2b and C2a. The complex formed by C4b and C2a is the CP C3 convertase, which is capable of processing C3 into C3a and C3b. The CP C5 convertase—C4b, C2a, C3b—is formed upon addition of a C3b monomer to the CP C3 convertase. (See, e.g., Müller-Eberhard (1988), supra and Cooper et al. (1970) J Exp Med 132:775-793.)

In addition to its role in C3 and C5 convertases, C3b also functions as an opsonin through its interaction with complement receptors present on the surfaces of antigen-presenting cells such as macrophages and dendritic cells. The opsonic function of C3b is generally considered to be one of the most important anti-infective functions of the complement system. Patients with genetic lesions that block C3b function are prone to infection by a broad variety of pathogenic organisms, while patients with lesions later in the complement cascade sequence, i.e., patients with lesions that block C5 functions, are found to be more prone only to Neisseria infection, and then only somewhat more prone.

The AP and CP C5 convertases cleave C5, which is a 190 kDa beta globulin found in normal human serum at approximately 75 μg/ml (0.4 μM). C5 is glycosylated, with about 1.5-3 percent of its mass attributed to carbohydrate. Mature C5 is a heterodimer of a 999 amino acid 115 kDa alpha chain that is disulfide linked to a 655 amino acid 75 kDa beta chain. C5 is synthesized as a single chain precursor protein product of a single copy gene (Haviland et al. (1991) J Immunol. 146:362-368). The cDNA sequence of the transcript of this gene predicts a secreted pro-05 precursor of 1658 amino acids along with an 18 amino acid leader sequence (see, e.g., U.S. Pat. No. 6,355,245).

The pro-05 precursor is cleaved after amino acids 655 and 659, to yield the beta chain as an amino terminal fragment (amino acid residues +1 to 655 of the above sequence) and the alpha chain as a carboxyl terminal fragment (amino acid residues 660 to 1658 of the above sequence), with four amino acids (amino acid residues 656-659 of the above sequence) deleted between the two.

C5a is cleaved from the alpha chain of C5 by either alternative or classical C5 convertase as an amino terminal fragment comprising the first 74 amino acids of the alpha chain (i.e., amino acid residues 660-733 of the above sequence). Approximately 20 percent of the 11 kDa mass of C5a is attributed to carbohydrate. The cleavage site for convertase action is at, or immediately adjacent to, amino acid residue 733 of the above sequence. A compound that would bind at, or adjacent, to this cleavage site would have the potential to block access of the C5 convertase enzymes to the cleavage site and thereby act as a complement inhibitor. A compound that binds to C5 at a site distal to the cleavage site could also have the potential to block C5 cleavage, for example, by way of steric hindrance-mediated inhibition of the interaction between C5 and the C5 convertase. A compound, in a mechanism of action consistent with that of the tick saliva complement inhibitor OmCI, may also prevent C5 cleavage by reducing flexibility of the C345C domain of the alpha chain of C5, which reduces access of the C5 convertase to the cleavage site of C5. See, e.g., Fredslund et al. (2008) Nat Immunol 9(7):753-760.

C5 can also be activated by means other than C5 convertase activity. Limited trypsin digestion (see, e.g., Minta and Man (1997) J Immunol 119:1597-1602 and Wetsel and Kolb (1982) J Immunol 128:2209-2216) and acid treatment (Yamamoto and Gewurz (1978) J Immunol 120:2008 and Damerau et al. (1989) molec Immunol 26:1133-1142) can also cleave C5 and produce active C5b.

Cleavage of C5 releases C5a, a potent anaphylatoxin and chemotactic factor, and leads to the formation of the lytic terminal complement complex, C5b-9. C5a and C5b-9 also have pleiotropic cell activating properties, by amplifying the release of downstream inflammatory factors, such as hydrolytic enzymes, reactive oxygen species, arachidonic acid metabolites and various cytokines

The first step in the formation of the terminal complement complex involves the combination of C5b with C6, C7, and C8 to form the C5b-8 complex at the surface of the target cell. Upon the binding of the C5b-8 complex with several C9 molecules, the membrane attack complex (MAC, C5b-9, terminal complement complex—TCC) is formed. When sufficient numbers of MACs insert into target cell membranes the openings they create (MAC pores) mediate rapid osmotic lysis of the target cells. Lower, non-lytic concentrations of MACs can produce other effects. In particular, membrane insertion of small numbers of the C5b-9 complexes into endothelial cells and platelets can cause deleterious cell activation. In some cases activation may precede cell lysis.

As mentioned above, C3a and C5a are anaphylatoxins. These activated complement components can trigger mast cell degranulation, which releases histamine from basophils and mast cells, and other mediators of inflammation, resulting in smooth muscle contraction, increased vascular permeability, leukocyte activation, and other inflammatory phenomena including cellular proliferation resulting in hypercellularity. C5a also functions as a chemotactic peptide that serves to attract pro-inflammatory granulocytes to the site of complement activation.

C5a receptors are found on the surfaces of bronchial and alveolar epithelial cells and bronchial smooth muscle cells. C5a receptors have also been found on eosinophils, mast cells, monocytes, neutrophils, and activated lymphocytes.

While a properly functioning complement system provides a robust defense against infecting microbes, inappropriate regulation or activation of complement has been implicated in the pathogenesis of a variety of disorders including, e.g., rheumatoid arthritis (RA); lupus nephritis; asthma; ischemia-reperfusion injury; atypical hemolytic uremic syndrome (aHUS); dense deposit disease (DDD); paroxysmal nocturnal hemoglobinuria (PNH); macular degeneration (e.g., age-related macular degeneration (AMD)); hemolysis, elevated liver enzymes, and low platelets (HELLP) syndrome; thrombotic thrombocytopenic purpura (TTP); spontaneous fetal loss; Pauci-immune vasculitis; epidermolysis bullosa; recurrent fetal loss; multiple sclerosis (MS); traumatic brain injury; and injury resulting from myocardial infarction, cardiopulmonary bypass and hemodialysis. (See, e.g., Holers et al. (2008) Immunological Reviews 223:300-316.) Inhibition of complement (e.g., inhibition of: terminal complement formation, C5 cleavage, or complement activation) has been demonstrated to be effective in treating several complement-associated disorders both in animal models and in humans. See, e.g., Rother et al. (2007) Nature Biotechnology 25(11):1256-1264; Wang et al. (1996) Proc Natl Acad Sci USA 93:8563-8568; Wang et al. (1995) Proc Natl Acad Sci USA 92:8955-8959; Rinder et al. (1995) J Clin Invest 96:1564-1572; Kroshus et al. (1995) Transplantation 60:1194-1202; Homeister et al. (1993) J Immunol 150:1055-1064; Weisman et al. (1990) Science 249:146-151; Amsterdam et al. (1995) Am J Physiol 268:H448-H457; and Rabinovici et al. (1992) J Immunol 149:1744 1750.

### SUMMARY

The disclosure is based, at least in part, on the discovery by the inventors that a single amino acid change in an anti-C5 single chain antibody, pexelizumab (Alexion Pharmaceuticals, Inc., Cheshire, Conn.), confers significant physico-chemical advantages to the antibody. (Pexelizumab, which is a single chain version of the whole antibody eculizumab, is described in detail in, e.g., Whiss (2002) Curr Opin Investig Drugs 3(6):870-7; Patel et al. (2005) Drugs Today (Banc) 41(3):165-70; Thomas et al. (1996) Mol Immunol 33(17-18):1389-401; and U.S. Pat. No. 6,355,245.) That is, by substituting the arginine (R) at position 38 (according to Kabat numbering and the amino acid sequence number set forth in SEQ ID NO:2) of the light chain of the pexelizumab antibody amino acid sequence with a glutamine (Q), the inventors observed, among other things, a dramatic change in the isoelectric point (pI) of the antibody. (See Kabat et al. (1991) “Sequences of Proteins of Immunological Interest.” NIH Publication No. 91-3242, U.S. Department of Health and Human Services, Bethesda, Md.) As predicted using sequence analysis software, the pI of pexelizumab is approximately 6.55, whereas the pI of the R38Q-substituted form of the antibody is 5.45. The R38Q-substituted antibody can be formulated in solution up to approximately 50 mg/mL at neutral pH, whereas pexelizumab reaches an upper limit of solubility at approximately 2 mg/mL. This indicates that the R38Q substitution confers a significant increase in the solubility of the antibody.

The increased solubility in aqueous solution of the R38Q-substituted antibody, as compared to the solubility of pexelizumab, is beneficial for several reasons. First, for therapeutic applications that require the antibody to be administered to a subject in a small volume (e.g., intraocular, intrapulmonary, intraarticular, or subcutaneous administration), therapeutic efficacy often turns on the amount of antibody that can be administered in that small volume. This therapeutic requirement necessitates formulation of the antibody at high concentrations, e.g., high concentration solutions. Second, high concentration antibody formulations can allow for more patient choice regarding the route of administration. For example, if intravenous infusion is used, a high concentration formulation allows for shorter infusion time. For therapeutic applications that require frequent and/or chronic administration, the subcutaneous route of delivery is made possible by high concentration formulations and can be more appealing to patients than intravenous infusion. Therefore, the ability to formulate the antibody at high concentrations can increase compliance of administration by providing an easy home administration alternative to patients with complement-associated disorders. Other benefits of high concentration formulations include, e.g., manufacturing cost savings from decreasing bulk storage space and/or the number of product fills.

As set forth in detail in the working examples, the R38Q substitution does not, however, significantly affect the affinity of the antibody for C5, nor does it significantly affect the activity of the antibody as both pexelizumab and the R38Q-substituted antibody prevent hemolysis of red blood cells when evaluated in a hemolytic assay.

Accordingly, the disclosure provides C5-binding polypeptides that have, inter alia, one or more of the aforementioned improved characteristics. The polypeptides are also capable of inhibiting, e.g., the cleavage of C5 to fragments C5a and C5b, and thus preventing the formation of terminal complement as well as the C5a-dependent inflammatory response. Thus, the C5-binding polypeptides described herein are also useful in a variety of diagnostic and therapeutic applications. For example, the polypeptides can be used to treat or prevent complement-associated conditions including, without limitation, paroxysmal nocturnal hemoglobinuria, atypical hemolytic uremic syndrome, age-related macular degeneration (e.g., wet or dry form AMD), graft rejection, rheumatoid arthritis, asthma, ischemia-reperfusion injury, atypical hemolytic uremic syndrome, thrombotic thrombocytopenic purpura, paroxysmal nocturnal hemoglobinuria, dense deposit disease, spontaneous fetal loss, Pauci-immune vasculitis, epidermolysis bullosa, recurrent fetal loss, multiple sclerosis, traumatic brain injury, myasthenia gravis (MG), cold agglutinin disease, dermatomyositis, Graves' disease, Hashimoto's thyroiditis, type I diabetes, psoriasis, pemphigus, autoimmune hemolytic anemia, idiopathic thrombocytopenic purpura, Goodpasture syndrome, multifocal motor neuropathy, neuromyelitis optica, antiphospholipid syndrome, Degos' disease, complement-associated pulmonary conditions (e.g., asthma and chronic obstructive pulmonary disease), catastrophic antiphospholipid syndrome, or any other complement-associated condition described herein and/or known in the art.

In one aspect, the disclosure features a polypeptide that binds to a human complement component C5 protein. The polypeptide can comprise, or consist of, the amino acid sequence depicted in SEQ ID NO:2. In some embodiments, a C5-binding polypeptide described herein is not a whole antibody. In some embodiments, a C5-binding polypeptide described herein is a single chain antibody.

“Polypeptide,” “peptide,” and “protein” are used interchangeably and mean any peptide-linked chain of amino acids, regardless of length or post-translational modification.

In another aspect, the disclosure features a C5-binding polypeptide that comprises an amino acid sequence that is greater than 50 (e.g., greater than or equal to 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99) % identical to the amino acid sequence depicted in SEQ ID NO:2, but contains the glutamine at position 38 of SEQ ID NO:2.

In another aspect, the disclosure features a polypeptide, which binds to human complement component C5 protein and comprises the amino acid sequence depicted in SEQ ID NO:2, but with not more than 30 (e.g., 29, 28, 27, 26, 25, 24, 23, 22, 21, 20, 19, 18, 17, 16, 15, 14, 13, 12, 11, 10, 9, 8, 7, 6, 5, 4, 3, 2, or 1) amino acid substitutions. The substitutions can be conservative or non-conservative. However, the polypeptide comprises the glutamine at position 38 of SEQ ID NO:2.

Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

In another aspect, the disclosure features a polypeptide that includes at least 20 (e.g., 22, 25, 27, 30, 32, 35, 37, 40, 42, 45, 47, 50, 52, 55, 57, 60, 62, 65, 67, 70, 72, 75, 77, 80, 82, 85, 87, 90, 92, 95, 97, 100, 105, 110, 115, 120, 125, 130, 135, 140, 145, 150, 155, 160, 165, 170, 175, 180, 185, 190, 195, or 200 or more) consecutive amino acids depicted in SEQ ID NO:2, wherein the amino acid sequence comprises the glutamine at position 38. In some embodiments, the polypeptide comprises at least 20, but fewer than 246 (e.g., 245, 244, 243, 242, 241, 240, 235, 230, 225, 220, 215, 210, 205, 200, 195, 190, 185, 180, 175, 170, 165, 160, 155, 150, 145, 140, 135, 130, 125, 120, 115, 110, 105, 100, 95, 90, or fewer) consecutive amino acids depicted in SEQ ID NO:2, wherein the amino acid sequence comprises the glutamine at position 38.

In some embodiments, the C5-binding polypeptides are deletion variants. Deletion variants can lack, e.g., one, two, three, four, five, six, seven, eight, nine, ten, 11, 12, 13, 14, 15, 16, 17, 18, 19, 20, 21, 22, 23, 24, 25, 30, 35, 40, 45, 50, 55, 60, 65, 70, 75, 80, or 90 or more single amino acids. Deletion variants can also lack one or more segments of two or more (e.g., two, three, four, five, six, seven, eight, nine, ten, 11, 12, 13, 14, 15, 16, 17, 18, 19, 20, 21, 22, 23, 24, 25, 30, 35, 40, 45, 50, 55, 60, 65, 70, 75, 80, or 90 or more) consecutive amino acids or non-contiguous single amino acids. Thus, in some embodiments, the deletion variants can comprise a first segment comprising amino acids 1-107 of SEQ ID NO:2 (inclusive of glutamine 38) and a second segment comprising amino acids 125-246 of SEQ ID NO:2. The two amino acid segments can be linked directly together or linked by an amino acid sequence that is heterologous to amino acids 1-107 and 125-246 of SEQ ID NO:2. For example, the heterologous amino acid sequence can be a linker sequence such as, but not limited to, a polyglycine or polyserine linker sequence described in, e.g., U.S. Pat. Nos. 5,525,491 and 5,258,498, the disclosures of each of which are incorporated herein by reference in their entirety. Additional polypeptide linkers are known in the art and described herein.

In some embodiments, a C5-binding polypeptide described herein can be a fusion protein. The fusion protein can comprise one or more C5-binding segments (e.g., C5-binding segments depicted in SEQ ID NO:2) and one or more segments that are heterologous to the C5-binding segment(s). The heterologous sequence can be, e.g., an antigenic tag (e.g., FLAG, polyhistidine, hemagglutinin (HA), glutathione-S-transferase (GST), or maltose-binding protein (MBP)). Heterologous sequences can also be proteins useful as diagnostic or detectable markers, for example, luciferase, green fluorescent protein (GFP), or chloramphenicol acetyl transferase (CAT). For example, the fusion protein can comprise a first segment comprising amino acids 1-107 of SEQ ID NO:2 (inclusive of glutamine 38) and a second segment comprising amino acids 125-246 of SEQ ID NO:2, wherein (i) the first and second segments are connected by a heterologous amino acid sequence, e.g., a heterologous linker amino acid sequence and/or (ii) the protein contains one or both of an amino-terminal and/or carboxy-terminal heterologous segment, e.g., a carboxy-terminal antigenic tag, an amino-terminal heterologous sequence encoding a detectable polypeptide, or any of the heterologous sequences described herein. In some embodiments, the heterologous sequence can be a targeting moiety that targets the C5-binding segment to a cell, tissue, or microenvironment of interest. In some embodiments, the targeting moiety is a soluble form of a human complement receptor (e.g., human complement receptor 2) or an antibody (e.g., a single chain antibody) that binds to C3b or C3d. In some embodiments, the targeting moiety is an antibody that binds to a tissue-specific antigen such as a kidney-specific antigen.

In another aspect, the disclosure features a construct comprising a C5-binding polypeptide described herein and a targeting moiety. The targeting moiety can be one that targets the C5-binding polypeptide to a site of complement activation such as, but not limited to, red blood cells (e.g., RBCs of patients afflicted with a hemolytic disease such as PNH), vasculature of a transplanted organ, an articulated joint, the lungs, or the eyes. In some embodiments, the targeting moiety is a soluble form of a complement receptor, e.g., a soluble form of human complement receptor 1 or human complement receptor 2. In some embodiments, the targeting moiety is an antibody. In such embodiments, the construct is a bispecific antibody. The targeting moiety can be an antibody that binds to C3b and/or C3d. In some embodiments, the targeting moiety can be an antibody that binds to a tissue-specific antigen such as a kidney specific antigen (e.g., KIM-1).

In some embodiments of any of the C5-binding polypeptides described herein, the polypeptides can inhibit the formation, and/or the activity, of terminal complement. For example, a C5-binding polypeptide can inhibit the cleavage of C5 into fragments C5a and C5b and thereby reduce subsequent deposition of C5b-9 on cells and the C5a-mediated inflammatory response.

In yet another aspect, the disclosure features a single-chain antibody that binds to human complement component C5 and has a solubility of between about 10 mg/mL and about 60 mg/mL in aqueous solution. In some embodiments, the single-chain antibody has a solubility of between about 20 mg/mL and about 50 mg/mL. In some embodiments, the single-chain antibody has a solubility of between about 40 mg/mL and about 55 mg/mL. In some embodiments, the single-chain antibody has a solubility of about 50 mg/mL. In some embodiments, the single-chain antibody comprises or consists of the amino acid sequence depicted in SEQ ID NO:2. In some embodiments, the single-chain antibody comprises an amino acid sequence that is greater than 50 (e.g., greater than or equal to 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99) % identical to the amino acid sequence depicted in SEQ ID NO:2. In some embodiments, the single-chain antibody comprises or consists of an amino acid sequence depicted in SEQ ID NO:2, but with not more than 20 (e.g., 20, 19, 18, 17, 16, 15, 14, 13, 12, 11, 10, 9, 8, 7, 6, 5, 4, 3, 2, or 1) amino acid substitutions.

In another aspect, the disclosure features: (i) a nucleic acid that encodes any of the C5-binding polypeptides described herein (e.g., variants, deletion variants, fragments, constructs, bispecific antibodies, or fusion proteins comprising amino acid sequences depicted in SEQ ID NO:2); (ii) a vector containing the nucleic acid; (iii) a cell comprising the nucleic acid or the vector; and (iv) methods for producing a polypeptide (e.g., any of the C5-binding polypeptides described herein) using the cell. The nucleic acid can contain, or consist of, the nucleotide sequence depicted in SEQ ID NO:1. In some embodiments, the nucleic acid can comprise or consist of nucleotides 1-738 of SEQ ID NO:1. The nucleic acid can optionally include a translation start sequence (ATG) or a translation termination sequence (e.g., TGA). The vector can include the nucleic acid operably linked to an expression control sequence. Such a vector can be referred to herein as an “expression vector.” The vector can be integrated into the genome of the cell or can be maintained within the cell as an episome. The cell can be, e.g., a prokaryotic cell or a eukaryotic cell. The cell can be, e.g., a bacterial cell, a fungal cell (e.g., a yeast cell), an insect cell, or a mammalian cell (e.g., a rabbit cell, a mouse cell, a rat cell, a hamster cell, a cat cell, a dog cell, a goat cell, a cow cell, a pig cell, a horse cell, or a non-human primate cell). In some embodiments, the cell is a human cell. In some embodiments, the cell is transformed or immortalized. In some embodiments, the cell is a primary cell. The method for producing the polypeptide (or fusion polypeptide) includes culturing the aforementioned cell under conditions suitable for expression of the polypeptide or fusion polypeptide by the cell. The method can also include isolating the polypeptide or fusion polypeptide from the cell or from the medium in which it was cultured.

In yet another aspect, the disclosure features a cell lysate containing any of the C5-binding polypeptides described herein. The lysate can be prepared from cells expressing the polypeptide.

In another aspect, the disclosure features a pharmaceutical composition containing any of the C5-binding polypeptides described herein and a pharmaceutically acceptable excipient, diluent, and/or carrier.

In another aspect, the disclosure features a stable, lyophilized composition comprising any of the C5-binding polypeptides described herein. In another aspect, the disclosure features a kit containing the lyophilized composition and an aqueous solution comprising a pharmaceutically acceptable excipient, diluent, and/or carrier, wherein the solution is for use in reconstituting the lyophilized composition for subsequent therapeutic administration to a human having, suspected of having, or at risk for developing, a complement-associated disorder.

In another aspect, the disclosure features a pharmaceutical solution containing any of the C5-binding polypeptides described herein, wherein the polypeptide is present (or formulated) in the solution at a concentration of between about 10 mg/mL to 100 mg/mL (e.g., between about 9 mg/mL and 90 mg/mL; between about 9 mg/mL and 50 mg/mL; between about 10 mg/mL and 50 mg/mL; between about 15 mg/mL and 50 mg/mL; between about 15 mg/mL and 110 mg/mL; between about 15 mg/mL and 100 mg/mL; between about 20 mg/mL and 100 mg/mL; between about 20 mg/mL and 80 mg/mL; between about 25 mg/mL and 100 mg/mL; between about 25 mg/mL and 85 mg/mL; between about 20 mg/mL and 50 mg/mL; between about 25 mg/mL and 50 mg/mL; between about 30 mg/mL and 100 mg/mL; between about 30 mg/mL and 50 mg/mL; between about 40 mg/mL and 100 mg/mL; between about 50 mg/mL and 100 mg/mL; or between about 20 mg/mL and 50 mg/mL). In some embodiments, the polypeptide is present in the solution at greater than (or at least or equal to) 10 (e.g., greater than, at least, or equal to: 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, or even 150) mg/mL. In some embodiments, the polypeptide is present in the solution at a concentration of about 50 mg/mL.

In yet another aspect, the disclosure provides a method for inhibiting the formation of terminal complement and/or C5a. The method includes contacting a biological sample with any of the C5-binding polypeptides described herein in an amount effective to inhibit the formation of terminal complement and/or C5a in the biological sample. The C5-binding polypeptide can be used in an amount that is effective to inhibit formation of terminal complement (or C5a) by at least 20 (e.g., 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 99) %. In some embodiments, the C5-binding polypeptide can be used in an amount that is effective to completely inhibit formation of the terminal complement (and/or C5a). The biological sample can be a blood sample, a serum sample, or a plasma sample. The biological sample can be one obtained from a subject (e.g., a human) having, suspected of having, or at risk for developing, a complement-associated disorder. In some embodiments, the method can include obtaining a biological sample from the subject.

In another aspect, the disclosure features a method for treating a complement-associated disorder, which method includes administering to a subject in need thereof any of the C5-binding polypeptides described herein in an amount effective to treat a complement-associated disorder in the subject. The C5-binding polypeptide can be administered to the subject in an amount and/or with a frequency effective to inhibit in the subject's serum formation of terminal complement (and/or C5a) by at least 20 (e.g., 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 99) %. In some embodiments, the C5-binding polypeptide can be administered in an amount and/or with a frequency effective to completely inhibit formation of the terminal complement (and/or C5a). In some embodiments, the C5-binding polypeptide can be administered to the subject in an amount and/or with a frequency effective to reduce serum complement activity in the subject to a level that is less than or equal to 50 (e.g., less than 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1) % of the level of complement activity in serum from a healthy patient (e.g., a patient that is not afflicted with a complement-associated disorder).

In some embodiments of any of the methods described herein, the complement-associated disorder can be an alternative complement pathway-associated disorder or a classical complement pathway associated disorder. The complement-associated disorder can be, e.g., paroxysmal nocturnal hemoglobinuria, atypical hemolytic uremic syndrome, typical hemolytic uremic syndrome, age-related macular degeneration, graft rejection, rheumatoid arthritis, a complement-associated pulmonary condition, ischemia-reperfusion injury, thrombotic thrombocytopenic purpura, paroxysmal nocturnal hemoglobinuria, dense deposit disease, age-related macular degeneration, spontaneous fetal loss, Pauci-immune vasculitis, epidermolysis bullosa, recurrent fetal loss, multiple sclerosis, traumatic brain injury, myasthenia gravis, cold agglutinin disease, dermatomyositis, Graves' disease, Hashimoto's thyroiditis, type I diabetes, psoriasis, pemphigus, autoimmune hemolytic anemia, idiopathic thrombocytopenic purpura, Goodpasture syndrome, multifocal motor neuropathy, neuromyelitis optica, antiphospholipid syndrome, catastrophic antiphospholipid syndrome, and any other complement-associated disorder described herein or known in the art of medicine. The graft rejection can be, e.g., kidney graft rejection, bone marrow graft rejection, skin graft rejection, heart graft rejection, lung graft rejection, or liver graft rejection. The pulmonary condition can be, e.g., asthma, bronchitis, a chronic obstructive pulmonary disease (COPD), interstitial lung diseases, lung malignancies, α-1 anti-trypsin deficiency, emphysema, bronchiectasis, bronchiolitis obliterans, sarcoidosis, pulmonary fibrosis, or a collagen vascular disorder.

In some embodiments of the methods described herein, the polypeptide is administered intravenously to the subject. In some embodiments of the methods described herein, the polypeptide is administered to the lungs of the subject. In some embodiments of the methods described herein, the polypeptide is administered to the subject by subcutaneous injection. In some embodiments of the methods described herein, the polypeptide is administered to the subject by way of intraarticular injection. In some embodiments of the methods described herein, the polypeptide is administered to the subject by way of intravitreal or intraocular injection. Additional routes of local administration (e.g., to the eye, an articulated joint, or the lungs of a subject) are described herein and known in the art. For example, in some embodiments of any of the methods described herein, a C5-binding polypeptide can be administered to the eye by way of a transscleral patch (see below).

In some embodiments, the methods described herein can include administering one or more additional therapeutic agents to the subject. The one or more additional therapeutic agents can be administered together as separate therapeutic compositions or one therapeutic composition can be formulated to include both: (i) one or more C5-binding polypeptides and (ii) one or more additional therapeutic agents. An additional therapeutic agent can be administered prior to, concurrently, or after administration of the C5-binding polypeptide. An additional agent and a C5-binding polypeptide can be administered using the same delivery method or route or the agent and polypeptide can be administered using different methods or routes. The additional therapeutic agents can be any of those described herein or known in the art as being useful for treating or preventing a complement-associated disorder.

In some embodiments of the methods described herein, the subject is a mammal. In some embodiments, the subject is a human. The subject can be, e.g., an infant or a female.

In yet another aspect, the disclosure features a conjugate comprising any of the C5-binding polypeptides described herein conjugated to a heterologous moiety. The heterologous moiety can be covalently or non-covalently conjugated to the polypeptide. The heterologous moiety can be a detectable label such as, e.g., an enzymatic label, a radioactive label, a fluorescent label, or a luminescent label. The heterologous moiety can be, e.g., a first member of a specific binding pair. For example, the heterologous moiety can be biotin, streptavidin, or an analog of biotin or streptavidin.

In another aspect, the disclosure features a method for treating or preventing a complement-associated pulmonary condition such as, but not limited to, asthma, bronchitis, a chronic obstructive pulmonary disease (COPD), interstitial lung diseases, lung malignancies, α-1 anti-trypsin deficiency, emphysema, bronchiectasis, bronchiolitis obliterans, sarcoidosis, pulmonary fibrosis, and a collagen vascular disorder. The methods include administering to a subject one or more of the C5-binding polypeptides described herein in an amount effective to treat or prevent the condition. The one or more C5-binding polypeptides can be, e.g., administered prior to manifestation of the pulmonary condition, during manifestation of the pulmonary condition, or after manifestation of the pulmonary condition. The one or more C5-binding polypeptides can be administered, e.g., intravenously, subcutaneously, or by way of intrapulmonary delivery. For example, the one or more C5-binding polypeptides can be delivered to the lungs of the subject by way of a nebulizer or inhaler. In some embodiments, the one or more C5-binding polypeptides are administered in conjunction with at least one (e.g., one, two, three, four, or five or more) additional agents useful for treating or preventing a complement-associated pulmonary disorder (e.g., ameliorating a symptom thereof). The at least one additional agent can be, e.g., a corticosteroid such as, but not limited to, dexamethasone. Other additional therapeutic agents suitable for use with the methods described herein are known in the art and set forth herein. The at least one additional active agent can be administered before, after, or concurrently with administration of the one or more C5-binding polypeptides. The at least one additional agent and one or more C5-binding polypeptides can be administered by the same delivery method or route. For example, an additional active agent and a C5-binding polypeptide can be administered by nebulizer. In some embodiments, an agent and C5-binding polypeptide are administered by different methods or routes. For example, a C5-binding polypeptide can be administered by infusion and an additional active agent can be administered by nebulizer.

In another aspect, the disclosure features a therapeutic kit containing one or more C5-binding polypeptides and means for intrapulmonary administration to a subject having, suspected of having, or at risk of developing, a complement-associated pulmonary disorder. The nebulizer can be, e.g., a jet air nebulizer, an ultrasonic nebulizer, a vibrating mesh nebulizer, or a shockwave nebulizer. The inhaler can be, e.g., a metered-dose inhaler (e.g., a pressurized metered dose inhaler). The composition can also optionally contain instructions for how to administer the C5-binding polypeptide(s) to a subject. The kit can also include one or more additional active agents for use in preventing or treating a complement-associated disorder in a subject.

In another aspect, the disclosure features a method for treating a complement-associated disorder of the eye such as, but not limited to, wet and/or dry AMD. The method includes administering to a subject afflicted with a complement-associated disorder of the eye a C5-binding polypeptide described herein in an amount and with a frequency to treat the disorder. The C5-binding polypeptide can be administered to the subject by way of intraocular or intravitreal administration. In some embodiments, the C5-binding polypeptide can be administered topically (e.g., formulated as an eye drop or as part of a soaking, hydrating, and/or cleansing solution for contact lenses) or by way of a transscleral patch. In some embodiments, the C5-binding polypeptide can be administered in conjunction with one or more additional therapeutic agents for treating a complement-associated disorder of the eye. For example, a C5-binding polypeptide described herein can be administered with a VEGF inhibitor (e.g., an antagonist anti-VEGF antibody such as bevacizumab, ranibizumab, pegaptanib sodium, or verteporfin (see below)). As described in detail below, the C5-binding polypeptide can be administered at the same time, prior to, or after the one or more additional therapeutic agents.

Percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2, or Megalign (DNASTAR) software. For consistency, the disclosure utilizes the BLAST software publicly available from the National Center of Biotechnology Information (U.S.). Appropriate parameters for measuring alignment, including any algorithms needed to achieve maximal alignment over the full-length of the sequences being compared can be determined by known methods.

Unless otherwise defined, all technical and scientific terms used herein have the same meaning as commonly understood by one of ordinary skill in the art to which this disclosure pertains. In case of conflict, the present document, including definitions, will control. Preferred methods and materials are described below, although methods and materials similar or equivalent to those described herein can also be used in the practice or testing of the presently disclosed methods and compositions. All publications, patent applications, patents, and other references mentioned herein are incorporated by reference in their entirety.

Other features and advantages of the present disclosure, e.g., methods for treating or preventing a complement-associated disorder, will be apparent from the following description, the examples, and from the claims.

### BRIEF DESCRIPTION OF THE DRAWINGS

FIG. 1 is a line graph depicting the concentration-dependent inhibition of chicken erythrocyte hemolysis by two single chain antibodies: pexelizumab (filled diamonds) and R38Q substituted form of pexelizumab (filled squares). The Y-axis represents the apparent absorbance at 415 nm as a measure of hemoglobin release. The X-axis represents the concentration (μg/mL) of each antibody.

FIG. 2 is a line graph depicting the concentration-dependent inhibition of chicken erythrocyte hemolysis by the R38Q substituted form of pexelizumab. The source of the R38Q substituted antibody used in the experiment was: (i) R38Q substituted antibody from a 50 mg/mL solution (filled diamonds); (ii) R38Q substituted antibody from a 10 mg/mL solution (filled squares); or (iii) R38Q substituted antibody from a 1.9 mg/mL solution (filled triangles). The Y-axis represents the apparent absorbance at 415 nm as a measure of hemoglobin release. The X-axis represents the concentration (μg/mL) of each antibody.

### DETAILED DESCRIPTION

The disclosure features polypeptides that bind to complement component C5 as well as nucleic acids that encode the polypeptides. The polypeptides can be used in a variety of diagnostic and therapeutic applications such as methods for treating or preventing complement-associated disorders. While in no way intended to be limiting, exemplary polypeptides, nucleic acids, conjugates, pharmaceutical compositions and formulations, and methods for using any of the foregoing are elaborated on below and are exemplified in the working Examples.

### Compositions

The compositions described herein contain one or more complement component C5-binding polypeptides. The polypeptides comprise single chain antibodies that specifically bind to C5. The C5-binding polypeptides can have an amino acid sequence that includes, or consists of, the following sequence:

(SEQ ID NO: 2)
DIQMTQSPSSLSASVGDRVTITCGASENIYGALNWYQQKPGKAPKLLI
YGATNLADGVPSRFSGSGSGTDFTLTISSLQPEDFATYYCQNVLNTPL
TFGQGTKVEIKRTGGGGSGGGGSGGGGSQVQLVQSGAEVKKPGASVKV
SCKASGYIFSNYWIQWVRQAPGQGLEWMGEILPGSGSTEYTENFKDRV
TMTRDTSTSTVYMELSSLRSEDTAVYYCARYFFGSSPNWYFDVWGQGT
LVTVSS.

As described in detail in the working examples, the single chain antibody having the amino acid sequence depicted in SEQ ID NO:2 is a variant of the single chain antibody pexelizumab in which the arginine (R) at position 38 has been substituted with a glutamine (Q). The R38Q substitution confers significant physico-chemical advantages to the variant antibody including, e.g., increased solubility in aqueous solution. The variant antibody contains: an antibody light chain variable region (amino acids 1-107 of SEQ ID NO:2); two amino acids of an immunoglobulin light chain constant region (amino acids 108 and 109); a flexible peptide linker (amino acids 110-124 of SEQ ID NO:2); and an antibody heavy chain variable region (amino acids 125-246 of SEQ ID NO:2).

In some embodiments, a C5-binding polypeptide comprises an amino acid sequence that is greater than at least 50 (e.g., greater than or equal to 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99) identical to the amino acid sequence depicted in SEQ ID NO:2. The amino acid sequence contains the glutamine at position 38 of SEQ ID NO:2. In some embodiments, the polypeptide comprises an amino acid sequence that is greater than at least 50% identical to the amino acid sequence depicted in SEQ ID NO:2, wherein the polypeptide comprises a first amino acid segment that is identical to amino acids 1-107 of SEQ ID NO:2 and a second segment that is identical to amino acids 125-246 of SEQ ID NO:2.

In some embodiments, a C5-binding polypeptide described herein is a variant polypeptide comprising the amino acid sequence depicted in SEQ ID NO:2, but with not more than 30 (e.g., 29, 28, 27, 26, 25, 24, 23, 22, 21, 20, 19, 18, 17, 16, 15, 14, 13, 12, 11, 10, 9, 8, 7, 6, 5, 4, 3, 2, or 1) amino acid substitutions. The substitutions can be conservative or non-conservative. However, the polypeptide must contain the glutamine at position 38 of SEQ ID NO:2. In some embodiments, the polypeptide contains no substitutions in amino acids 1-107 of SEQ ID NO:2 and/or no substitutions in amino acids 125-246 of SEQ ID NO:2.

In some embodiments, the C5-binding polypeptide comprises a fragment of a polypeptide having at least 50% (see above) sequence identity with the amino acid sequence depicted in SEQ ID NO:2 or a fragment of a variant polypeptide described above. For example, a C5-binding polypeptide can include at least 20 (e.g., 22, 25, 27, 30, 32, 35, 37, 40, 42, 45, 47, 50, 52, 55, 57, 60, 62, 65, 67, 70, 72, 75, 77, 80, 82, 85, 87, 90, 92, 95, 97, 100, 105, 110, 115, 120, 125, 130, 135, 140, 145, 150, 155, 160, 165, 170, 175, 180, 185, 190, 195, or 200 or more) consecutive amino acids depicted in SEQ ID NO:2, wherein the amino acid sequence comprises the glutamine at position 38 of SEQ ID NO:2. In some embodiments, the polypeptide comprises at least 20, but fewer than 246 (e.g., 245, 244, 243, 242, 241, 240, 235, 230, 225, 220, 215, 210, 205, 200, 195, 190, 185, 180, 175, 170, 165, 160, 155, 150, 145, 140, 135, 130, 125, 120, 115, 110, 105, 100, 95, 90, or fewer) consecutive amino acids depicted in SEQ ID NO:2, wherein the amino acid sequence comprises the glutamine at position 38 of SEQ ID NO:2. All that is required of the fragment polypeptide is that it binds to complement component C5.

In some embodiments, the C5-binding polypeptides are deletion variants, which retain the glutamine at position 38 of SEQ ID NO:2. As described above, deletion variants can lack, e.g., one, two, three, four, five, six, seven, eight, nine, ten, 11, 12, 13, 14, 15, 16, 17, 18, 19, 20, 21, 22, 23, 24, 25, 30, 35, 40, 45, 50, 55, 60, 65, 70, 75, 80, or 90 or more single amino acids. Deletion variants can also lack one or more segments of two or more (e.g., two, three, four, five, six, seven, eight, nine, ten, 11, 12, 13, 14, 15, 16, 17, 18, 19, 20, 21, 22, 23, 24, 25, 30, 35, 40, 45, 50, 55, 60, 65, 70, 75, 80, or 90 or more) consecutive amino acids or non-contiguous single amino acids. The deletion can occur at the carboxy-terminus and/or amino-terminus of the polypeptide. In some embodiments, the deletion can be an internal deletion. For example, a C5-binding deletion variant polypeptide can comprise a first segment comprising amino acids 1-107 of SEQ ID NO:2 (inclusive of glutamine 38) and a second segment comprising amino acids 125-246 of SEQ ID NO:2. The two amino acid segments can be linked directly together or linked by an amino acid sequence that is heterologous to the first and second segments. In some embodiments, the heterologous amino acid sequence can be a polyglycine or polyserine linker moiety described in, e.g., U.S. Pat. Nos. 5,525,491 and 5,258,498, the disclosures of each of which are incorporated herein by reference in their entirety. In some embodiments, the heterologous amino acid sequence comprises, or consists of, GGGGSGGGGSGGGGS (SEQ ID NO:3).

In some embodiments, a C5-binding polypeptide described herein can be a fusion protein. The fusion protein can comprise one or more C5-binding segments (e.g., segments of the amino acid sequence depicted in SEQ ID NO:2) and one or more segments that are heterologous to the C5-binding segment(s). The heterologous sequence can be, e.g., an antigenic tag (e.g., FLAG, polyhistidine, hemagglutinin (HA), glutathione-S-transferase (GST), or maltose-binding protein (MBP)). Heterologous sequences can also be proteins useful as diagnostic or detectable markers, for example, luciferase, green fluorescent protein (GFP), or chloramphenicol acetyl transferase (CAT). For example, the fusion protein can comprise a first segment comprising amino acids 1-107 of SEQ ID NO:2 (inclusive of glutamine 38) and a second segment comprising amino acids 125-246 of SEQ ID NO:2, wherein the first and second segments are connected by a heterologous amino acid sequence. In another example, the fusion protein can comprise a C5-binding segment comprising amino acids 1-246 of SEQ ID NO:2 and an amino-terminal and/or carboxy-terminal heterologous segment, e.g., a carboxy-terminal antigenic tag.

In some embodiments, the C5-binding polypeptides described herein can comprise (e.g., as a fusion protein) or be joined with (e.g., chemically joined to) a heterologous moiety that targets the polypeptides to a site of complement activation, e.g., the surface of red blood cells (e.g., red blood cells in a PNH patient), the kidney (e.g., a transplanted kidney), an articulated joint (e.g., a joint of a patient with rheumatoid arthritis), or the eye (e.g., the macula).

The C5-binding polypeptides described herein specifically bind to a human complement component C5 protein (e.g., the human C5 protein having the amino acid sequence depicted in SEQ ID NO:4). The terms “specific binding” or “specifically binds” refer to two molecules forming a complex (e.g., a complex between a C5-binding polypeptide and a complement component C5 protein) that is relatively stable under physiologic conditions. Typically, binding is considered specific when the association constant (kₐ) is higher than 10⁶ M⁻¹s⁻¹. In some embodiments, a C5-binding polypeptide described herein has a dissociation constant (k_{d}) of less than or equal to 10⁻³ (e.g., 8×10⁻⁴, 5×10⁻⁴, 2×10⁻⁴, 10⁻⁴, or 10⁻⁵) s⁻¹. In some embodiments, a C5-binding polypeptide described herein has a K_{D} of less than 10⁻⁸, 10⁻⁹, 10⁻¹⁰, 10⁻¹¹, or 10⁻¹² M. The equilibrium constant K_{D} is the ratio of the kinetic rate constants—k_{d}/kₐ. In some embodiments, a C5-binding polypeptide described herein has a K_{D} of less than 1×10⁻⁹ M (e.g., less than 1×10⁻¹⁰ M).

Methods for determining whether a C5-binding polypeptide binds to a C5 protein and/or the affinity of the C5-binding polypeptide for a C5 protein are known in the art. For example, the interaction between a C5-binding polypeptide and C5 can be detected and/or quantified using a variety of techniques such as, but not limited to, Western blot, dot blot, plasmon surface resonance method (e.g., Biacore system; Pharmacia Biosensor AB, Uppsala, Sweden and Piscataway, N.J.), Octet, or enzyme-linked immunosorbent assay (ELISA) assays. See, e.g., Harlow and Lane (1988) “Antibodies: A Laboratory Manual” Cold Spring Harbor Laboratory Press, Cold Spring Harbor, N.Y.; Benny K. C. Lo (2004) “Antibody Engineering: Methods and Protocols,” Humana Press (ISBN: 1588290921); Borrebaek (1992) “Antibody Engineering, A Practical Guide,” W.H. Freeman and Co., NY; Borrebaek (1995) “Antibody Engineering,” 2^{nd} Edition, Oxford University Press, NY, Oxford; Johne et al. (1993) J Immunol Meth 160:191-198; Jonsson et al. (1993) Ann Biol Clin 51:19-26; and Jonsson et al. (1991) Biotechniques 11:620-627. See also U.S. Pat. No. 6,355,245.

As described above, the presently disclosed C5-binding polypeptides can inhibit complement component C5. In particular, the polypeptides inhibit the generation of the C5a anaphylotoxin and/or C5b active fragments of a complement component C5 protein (e.g., a human C5 protein). Accordingly, the C5-binding polypeptides inhibit, e.g., the pro-inflammatory effects of C5a and the generation of the C5b-9 membrane attack complex (MAC) at the surface of a cell and subsequent cell lysis. (See, e.g., Moongkarndi et al. (1982) Immunobiol 162:397 and Moongkarndi et al. (1983) Immunobiol 165:323.)

Suitable methods for measuring inhibition of C5 cleavage are described herein and are known in the art. For example, the concentration and/or physiologic activity of C5a and C5b in a body fluid can be measured by methods well known in the art. Methods for measuring C5a concentration or activity include, e.g., chemotaxis assays, RIAs, or ELISAs (see, e.g., Ward and Zvaifler (1971) J Clin Invest 50(3):606-16 and Wurzner et al. (1991) Complement Inflamm 8:328-340). For C5b, hemolytic assays or assays for soluble C5b-9 as discussed herein can be used. Other assays known in the art can also be used.

Inhibition of complement component C5 can also reduce the cell-lysing ability of complement in a subject's body fluids. Such reductions of the cell-lysing ability of complement present can be measured by methods well known in the art such as, for example, by a conventional hemolytic assay such as the hemolysis assay described by Kabat and Mayer (eds), “Experimental Immunochemistry, 2^{nd} Edition,” 135-240, Springfield, Ill., CC Thomas (1961), pages 135-139, or a conventional variation of that assay such as the chicken erythrocyte hemolysis method as described in, e.g., Hillmen et al. (2004) N Engl J Med 350(6):552.

The C5-binding polypeptides described herein can be produced using a variety of techniques known in the art of molecular biology and protein chemistry. For example, a nucleic acid encoding a C5-binding polypeptide described herein (e.g., a C5-binding polypeptide comprising or consisting of the amino acid sequence depicted in SEQ ID NO:2) can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system such that it can be maintained in two different organisms, for example in mammalian or insect cells for expression and in a prokaryotic host for cloning and amplification. An exemplary nucleic acid, which encodes an exemplary C5-binding polypeptide, is as follows:

(SEQ ID NO: 1)
GATATCCAGATGACCCAGTCCCCGTCCTCCCTGTCCGCCTCTGTGGGC
GATAGGGTCACCATCACCTGCGGCGCCAGCGAAAACATCTATGGCGCG
CTGAACTGGTATCAACAGAAACCCGGGAAAGCTCCGAAGCTTCTGATT
TACGGTGCGACGAACCTGGCAGATGGAGTCCCTTCTCGCTTCTCTGGA
TCCGGCTCCGGAACGGATTTCACTCTGACCATCAGCAGTCTGCAGCCT
GAAGACTTCGCTACGTATTACTGTCAGAACGTTTTAAATACTCCGTTG
ACTTTCGGACAGGGTACCAAGGTGGAAATAAAACGTACTGGCGGTGGT
GGTTCTGGTGGCGGTGGATCTGGTGGTGGCGGTTCTCAAGTCCAACTG
GTGCAATCCGGCGCCGAGGTCAAGAAGCCAGGGGCCTCAGTCAAAGTG
TCCTGTAAAGCTAGCGGCTATATTTTTTCTAATTATTGGATTCAATGG
GTGCGTCAGGCCCCCGGGCAGGGCCTGGAATGGATGGGTGAGATCTTA
CCGGGCTCTGGTAGCACCGAATATACCGAAAATTTTAAAGACCGTGTT
ACTATGACGCGTGACACTTCGACTAGTACAGTATACATGGAGCTCTCC
AGCCTGCGATCGGAGGACACGGCCGTCTATTATTGCGCGCGTTATTTT
TTTGGTTCTAGCCCGAATTGGTATTTTGATGTTTGGGGTCAAGGAACC
CTGGTCACTGTCTCGAGCTGA.

In some embodiments, the nucleic acid comprises nucleotides 1-738 of SEQ ID NO:1, e.g., in embodiments where carboxy-terminal fusion proteins are to be generated or produced.

Several possible vector systems are available for the expression of C5-binding polypeptides from nucleic acids in mammalian cells. One class of vectors relies upon the integration of the desired gene sequences into the host cell genome. Cells which have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet. 1:327). The selectable marker gene can be either linked to the DNA gene sequences to be expressed, or introduced into the same cell by co-transfection (Wigler et al. (1979) Cell 16:77). A second class of vectors utilizes DNA elements which confer autonomously replicating capabilities to an extrachromosomal plasmid. These vectors can be derived from animal viruses, such as bovine papillomavirus (Sarver et al. (1982) Proc Natl Acad Sci USA, 79:7147), polyoma virus (Deans et al. (1984) Proc Natl Acad Sci USA 81:1292), or SV40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, lipofectin, electroporation, viral infection, dextran-mediated transfection, polybrene-mediated transfection, protoplast fusion, and direct microinjection.

Appropriate host cells for the expression of the C5-binding polypeptides include yeast, bacteria, insect, plant, and mammalian cells. Of particular interest are bacteria such as E. coli, fungi such as Saccharomyces cerevisiae and Pichia pastoris, insect cells such as SF9, mammalian cell lines (e.g., human cell lines), as well as primary cell lines (e.g., primary mammalian cells). In some embodiments, the C5-binding polypeptides can be expressed in Chinese hamster ovary (CHO) cells or in a suitable myeloma cell line such as (NSO).

In some embodiments, a C5-binding polypeptide can be expressed in, and purified from, transgenic animals (e.g., transgenic mammals). For example, a C5-binding polypeptide can be produced in transgenic non-human mammals (e.g., rodents, sheep or goats) and isolated from milk as described in, e.g., Houdebine (2002) Curr Opin Biotechnol 13(6):625-629; van Kuik-Romeijn et al. (2000) Transgenic Res 9(2):155-159; and Pollock et al. (1999) J Immunol Methods 231(1-21:147-157.

The C5-binding polypeptides described herein can be produced from cells by culturing a host cell transformed with the expression vector containing nucleic acid encoding the antibodies, under conditions, and for an amount of time, sufficient to allow expression of the proteins. Such conditions for protein expression will vary with the choice of the expression vector and the host cell, and will be easily ascertained by one skilled in the art through routine experimentation. For example, polypeptides expressed in E. coli can be refolded from inclusion bodies (see, e.g., Hou et al. (1998) Cytokine 10:319-30). Bacterial expression systems and methods for their use are well known in the art (see Current Protocols in Molecular Biology, Wiley & Sons, and Molecular Cloning—A Laboratory Manual—3rd Ed., Cold Spring Harbor Laboratory Press, New York (2001)). The choice of codons, suitable expression vectors and suitable host cells will vary depending on a number of factors, and may be easily optimized as needed. A C5-binding polypeptide described herein can be expressed in mammalian cells or in other expression systems including but not limited to yeast, baculovirus, and in vitro expression systems (see, e.g., Kaszubska et al. (2000) Protein Expression and Purification 18:213-220).

Following expression, the C5-binding polypeptides can be isolated. The term “purified” or “isolated” as applied to any of the proteins described herein (e.g., a C5-binding polypeptide) refers to a polypeptide that has been separated or purified from components (e.g., proteins or other naturally-occurring biological or organic molecules) which naturally accompany it, e.g., other proteins, lipids, and nucleic acid in a prokaryote expressing the proteins. Typically, a polypeptide is purified when it constitutes at least 60 (e.g., at least 65, 70, 75, 80, 85, 90, 92, 95, 97, or 99) %, by weight, of the total protein in a sample.

A C5-binding polypeptide can be isolated or purified in a variety of ways known to those skilled in the art depending on what other components are present in the sample. Standard purification methods include electrophoretic, molecular, immunological, and chromatographic techniques, including ion exchange, hydrophobic, affinity, and reverse-phase HPLC chromatography. For example, a C5-binding polypeptide can be purified using a standard anti-antibody column or, e.g., a protein-A or protein-G column. Ultrafiltration and diafiltration techniques, in conjunction with protein concentration, are also useful. See, e.g., Scopes (1994) “Protein Purification, 3^{rd} edition,” Springer-Verlag, New York City, N.Y. The degree of purification necessary will vary depending on the desired use. In some instances, no purification of the expressed polypeptide thereof will be necessary.

Methods for determining the yield or purity of a purified polypeptide are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

In some embodiments, endotoxin can be removed from the C5-binding polypeptide preparations. Methods for removing endotoxin from a protein sample are known in the art. For example, endotoxin can be removed from a protein sample using a variety of commercially available reagents including, without limitation, the ProteoSpin™ Endotoxin Removal Kits (Norgen Biotek Corporation), Detoxi-Gel Endotoxin Removal Gel (Thermo Scientific; Pierce Protein Research Products), MiraCLEAN® Endotoxin Removal Kit (Minis), or Acrodisc™—Mustang® E membrane (Pall Corporation).

Methods for detecting and/or measuring the amount of endotoxin present in a sample (both before and after purification) are known in the art and commercial kits are available. For example, the concentration of endotoxin in a protein sample can be determined using the QCL-1000 Chromogenic kit (BioWhittaker), the limulus amebocyte lysate (LAL)-based kits such as the Pyrotell®, Pyrotell®-T, Pyrochrome®, Chromo-LAL, and C5E kits available from the Associates of Cape Cod Incorporated.

Conjugates and Fusion Proteins

The C5-binding polypeptides can be modified following their expression and purification. The modifications can be covalent or non-covalent modifications. Such modifications can be introduced into the C5-binding polypeptides by, e.g., reacting targeted amino acid residues of the polypeptide with an organic derivatizing agent that is capable of reacting with selected side chains or terminal residues. Suitable sites for modification can be chosen using any of a variety of criteria including, e.g., structural analysis or amino acid sequence analysis of the C5-binding polypeptides.

In some embodiments, the C5-binding polypeptides can be conjugated to a heterologous moiety. In embodiments where the heterologous moiety is a polypeptide, a C5-binding polypeptide and heterologous moiety described herein can be joined by way of fusion protein. The heterologous moiety can be, e.g., a heterologous polypeptide, a therapeutic agent (e.g., a toxin or a drug), or a detectable label such as, but not limited to, a radioactive label, an enzymatic label, a fluorescent label, or a luminescent label. Suitable heterologous polypeptides include, e.g., an antigenic tag (e.g., FLAG, polyhistidine, hemagglutinin (HA), glutathione-S-transferase (GST), or maltose-binding protein (MBP)) for use in purifying the antibodies. Heterologous polypeptides also include polypeptides that are useful as diagnostic or detectable markers, for example, luciferase, green fluorescent protein (GFP), or chloramphenicol acetyl transferase (CAT). Where the heterologous moiety is a polypeptide, the moiety can be incorporated into a C5-binding polypeptide, resulting in a fusion protein. Heterologous polypeptides also include, e.g., growth factors, cytokines, and chemokines Growth factors can include, e.g., vascular endothelial growth factor (VEGF), insulin-like growth factor (IGF), bone morphogenic protein (BMP), granulocyte-colony stimulating factor (G-C5F), granulocyte-macrophage colony stimulating factor (GM-C5F), nerve growth factor (NGF); a neurotrophin, platelet-derived growth factor (PDGF), erythropoietin (EPO), thrombopoietin (TPO), myostatin (GDF-8), growth differentiation factor-9 (GDF9), basic fibroblast growth factor (bFGF or FGF2), epidermal growth factor (EGF), hepatocyte growth factor (HGF), and a neuregulin (e.g., NRG1, NRG2, NRG3, or NRG4). Cytokines include, e.g., interferons (e.g., IFNγ), tumor necrosis factor (e.g., TNFα or TNFβ), and the interleukins (e.g., IL-1 to IL-33 (e.g., IL-1, IL-2, IL-3, IL-4, IL-5, IL-6, IL-7, IL-8, IL-9, IL-10, IL-12, IL-13, or IL-15)). Chemokines include, e.g., I-309, TCA-3, MCP-I, MIP-1α, MIP-1β, RANTES, C10, MRP-2, MARC, MCP-3, MCP-2, MRP-2, CCF18, Eotaxin, MCP-5, MCP-4, NCC-I, HCC-I, leukotactin-1, LEC, NCC-4, TARC, PARC, or Eotaxin-2. In some embodiments, the heterologous moiety is a targeting moiety.

The disclosure also features a construct comprising a C5-binding polypeptide described herein and a targeting moiety that targets the C5-binding polypeptide to a cell, tissue, or biological microenvironment of interest. For example, a construct can contain a C5-binding polypeptide and a targeting moiety that targets the polypeptide to a site of complement activation (e.g., red blood cells of patients with hemolytic disease such as PNH, CAD, aHUS, or TTP). The site of complement activation can also be, e.g., the vasculature of a transplanted organ, the eye of a patient with AMD, the lungs of a patient with asthma or COPD, or an articulated joint of a patient afflicted with RA. Such targeting moieties can include, e.g., soluble form of complement receptor 1 (CR1), a soluble form of complement receptor 2 (CR2), or an antibody (or antigen-binding fragment thereof) that binds to C3b and/or C3d. Methods for generating fusion proteins (e.g., fusion proteins containing a C5-binding polypeptide and a soluble form of human CR1 or human CR2) are known in the art and described in, e.g., U.S. Pat. No. 6,897,290; U.S. patent application publication no. 2005265995; and Song et al. (2003) J Clin Invest 11(12):1875-1885. Methods for producing a bispecific antibody (e.g., a bispecific antibody comprising a C5-binding polypeptide described herein and an antibody that binds to C3b and/or C3d) are also known in the art and described herein.

In some embodiments, a C5-binding polypeptide can contain a moiety that targets the polypeptide to the kidney. Such constructs can be useful, e.g., for treating complement-associated diseases of the kidney such as, but not limited to, renal ischemia-reperfusion injury (IRI), renal transplant rejection, or hemolytic uremic syndrome. Antigens to which a kidney targeting moiety can bind include, e.g., dipeptidylpeptidase IV (DPPIV), Lrp2 (megalin), Cubn (cubilin), Abcc2 (ATP binding cassette, sub-family C, member 2), Abcc4 (ATP binding cassette, sub-family C, member 4), Abcb1b (ATP binding cassette, sub-family B, member 1; P-glycoprotein), Slc1a1 (excitatory amino acid carrier 1), Slc3a1 (cystine, dibasic and neutral amino acid transporters), SlcSa1 (sodium/glucose cotransporter 1), Slc5a2 (sodium/glucose cotransporter 2), Slc9a3 (sodium/hydrogen exchanger 3), Slc10a2 (sodium/taurocholate cotransporting polypeptide), Slc13a2 (sodium dependent dicarboxylate cotransporter), Sic15a 1 (oligopeptide transporter 1), Sic15a2 (oligopeptide transporter 2), Slc17a1 (sodium phosphate transporter 1), Slc17a2 (sodium phosphate transporter 3), Slc17a3 (sodium phosphate transporter 4), Slco1a1 (organic anion transporter protein 1), Slc22a4 (organic cation transporter OCTN1), Slc22a5 (organic cation transporter OCTN2), Slc22a11 (organic anion transporter 4), Slc34a1 (sodium phosphate transporter IIa), megalin (low density lipoprotein receptor-related protein 2, LRP2), neutral endopeptidase (NEP), CD10, mucin 20 (or other mucins), kidney-injury molecule 1 (KIM-1), or hepatitis A virus cellular receptor 1 and megalin.

A wide variety of bispecific antibody formats are known in the art of antibody engineering and methods for making the bispecific antibodies (e.g., a bispecific antibody comprising a C5-binding polypeptide described herein and an antibody that binds to C3b, C3d, or a tissue-specific antigen) are well within the purview of those skilled in the art. Traditionally, the recombinant production of bispecific antibodies is based on the co-expression of two immunoglobulin heavy-chain/light-chain pairs, where the two heavy chains have different specificities (Milstein and Cuello (1983) Nature 305:537-539). Antibody variable domains with the desired binding specificities (antibody-antigen combining sites) can be fused to immunoglobulin constant domain sequences. The fusion can include an immunoglobulin heavy-chain constant domain, e.g., at least part of the hinge, CH2, and CH3 regions. DNAs encoding the immunoglobulin heavy-chain fusions and, if desired, the immunoglobulin light chain, are inserted into separate expression vectors, and are co-transfected into a suitable host organism. For further details of illustrative currently known methods for generating bispecific antibodies see, e.g., Suresh et al. (1986) Methods in Enzymology 121:210; PCT Publication No. WO 96/27011; Brennan et al. (1985) Science 229:81; Shalaby et al., J. Exp. Med. (1992) 175:217-225; Kostelny et al. (1992) J Immunol 148(5):1547-1553; Hollinger et al. (1993) Proc Natl Acad Sci USA 90:6444-6448; Gruber et al. (1994) J Immunol 152:5368; and Tutt et al. (1991) J Immunol 147:60. Bispecific antibodies also include cross-linked or heteroconjugate antibodies. Heteroconjugate antibodies may be made using any convenient cross-linking methods. Suitable cross-linking agents are well known in the art, and are disclosed in U.S. Pat. No. 4,676,980, along with a number of cross-linking techniques.

U.S. Pat. No. 5,534,254 describes several different types of bispecific antibodies including, e.g., single chain Fv fragments linked together by peptide couplers, chelating agents, or chemical or disulfide couplings. In another example, Segal and Bast [(1995) Curr Protocols Immunol Suppl. 14:2.13.1-2.13.16] describes methods for chemically cross-linking two monospecific antibodies to thus form a bispecific antibody. As described above, a bispecific antibody described herein can be formed, e.g., by conjugating two single chain antibodies which are selected from, e.g., a C5-binding polypeptide described herein and an antibody that binds to, e.g., C3b, C3d, or a lung-specific antigen, an eye-specific antigen, or a kidney-specific antigen.

Various techniques for making and isolating bispecific antibody fragments directly from recombinant cell culture have also been described. For example, bispecific antibodies have been produced using leucine zippers. (See, e.g., Kostelny et al. (1992) J Immunol 148(5):1547-1553 and de Kruif and Logtenberg (1996) J Biol Chem 271(13):7630-7634.) The leucine zipper peptides from the Fos and Jun proteins may be linked to the Fab′ portions of two different antibodies by gene fusion. The antibody homodimers may be reduced at the hinge region to form monomers and then re-oxidized to form the antibody heterodimers.

In some embodiments, the bispecific antibody can be a tandem single chain (sc) Fv fragment, which contains two different scFv fragments covalently tethered together by a linker (e.g., a polypeptide linker). See, e.g., Ren-Heidenreich et al. (2004) Cancer 100:1095-1103 and Korn et al. (2004) J Gene Med 6:642-651. Examples of linkers can include but are not limited to (Gly₄Ser)₂, (Gly₄Ser)₃ (G₄5), (Gly₃Ser)₄(G₃S), SerGly₄, and SerGly₄SerGly₄. In some embodiments, the linker can contain, or be, all or part of a heavy chain polypeptide constant region such as a CH1 domain as described in, e.g., Grosse-Hovest et al. (2004) Proc Natl Acad Sci USA 101:6858-6863. In some embodiments, the two antibody fragments can be covalently tethered together by way of a polyglycine-serine or polyserine-glycine linker as described in, e.g., U.S. Pat. Nos. 7,112,324 and 5,525,491, respectively. See also U.S. Pat. No. 5,258,498, the disclosure with respect to antibody engineering and linkers is incorporated herein by reference in its entirety. Methods for generating bispecific tandem scFv antibodies are described in, e.g., Maletz et al. (2001) Int J Cancer 93:409-416; Hayden et al. (1994) Ther Immunol 1:3-15; and Honemann et al. (2004) Leukemia 18:636-644. Alternatively, the antibodies can be “linear antibodies” as described in, e.g., Zapata et al. (1995) Protein Eng. 8(10):1057-1062. Briefly, these antibodies comprise a pair of tandem Fd segments (V_{H}-C_{H}1-V_{H}-C_{H}1) that form a pair of antigen binding regions.

A bispecific antibody can also be a diabody. Diabody technology described by, e.g., Hollinger et al. (1993) Proc Natl Acad Sci USA 90:6444-6448 has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. (See also, e.g., Zhu et al. (1996) Biotechnology 14:192-196 and Helfrich et al. (1998) Int J Cancer 76:232-239.) Bispecific single chain diabodies (scDb) as well as methods for generating scDb are described in, e.g., Brüsselbach et al. (1999) Tumor Targeting 4:115-123; Kipriyanov et al. (1999) J Mol Biol 293:41-56; and Nettlebeck et al. (2001) Mol Ther 3:882-891.

The disclosure also embraces variant forms of bispecific antibodies such as the tetravalent dual variable domain immunoglobulin (DVD-Ig) molecules described in Wu et al. (2007) Nat Biotechnol 25(11):1290-1297. The DVD-Ig molecules are designed such that two different light chain variable domains (VL) from two different parent antibodies are linked in tandem directly or via a short linker by recombinant DNA techniques, followed by the light chain constant domain. Methods for generating DVD-Ig molecules from two parent antibodies are further described in, e.g., PCT Publication Nos. WO 08/024,188 and WO 07/024,715, the disclosures of each of which are incorporated herein by reference in their entirety. Also embraced is the bispecific format described in, e.g., U.S. patent application publication no. 20070004909, the disclosure of which is incorporated by reference in its entirety.

Exemplary anti-C3b antibodies as well as methods suitable for producing such antibodies are well known in the art and described in, e.g., PCT publication no. WO 87/06344; U.S. Pat. No. 6,572,856; Peng et al. (2004) J Clin Oncol 22(145):2621; and Peng et al. (2005) Cancer Immunol Immunother 54(12):1172-9, the disclosures of each of which are incorporated herein by reference in their entirety. Exemplary anti-C3d antibodies as well as methods suitable for producing such antibodies are well known in the art and described in, e.g., Cruz and Leon (2007) Hybridoma 26(6):433-4; Koistinen et al. (1989) Complement Inflamm 6(4):270-280; and Dobbie et al. (1987) Transfusion 27(6):453-459, the disclosures of each of which are incorporated herein by reference in their entirety.

The C5-binding polypeptides and targeting-moieties that are used to form the bispecific antibody molecules described herein can be, e.g., chimeric, humanized, rehumanized, deimmunized, or fully human. Chimeric antibodies are produced by recombinant processes well known in the art of antibody engineering and have a non-human mammal variable region and a human constant region. Humanized antibodies correspond more closely to the sequence of human antibodies than do chimeric antibodies. Humanized variable domains are constructed in which amino acid sequences of one or more CDRs of non-human origin are grafted to human framework regions (FRs) as described in, e.g., Jones et al. (1996) Nature 321: 522-525; Riechmann et al. (1988) Nature 332:323-327 and U.S. Pat. No. 5,530,101. The humanized antibody can be an antibody that contains one or more human framework regions that are not germline. For example, the humanized antibody can contain one or more framework regions that were subject to somatic hypermutation and thus no longer germline per se. (See, e.g., Abbas, Lichtman, and Pober (2000) “Cellular and Molecular Immunology,” 4^{th} Edition, W.B. Saunders Company (ISBN:0721682332)). In some embodiments, the humanized antibody contains human germline framework regions, e.g., human germline V_{H} regions, human germline D regions, and human germline J regions (e.g., human germline J_{H} regions). The MRC Center for Protein Engineering maintains the online VBase database system, which includes amino acid sequences for a large number of human germline framework regions. See, e.g., Welschof et al. (1995) J Immunol Methods 179:203-214; Chothia et al. (1992) J Mol Biol 227:776-798; Williams et al. (1996) J Mol Biol 264:220-232; Marks et al. (1991) Eur J Immunol 21:985-991; and Tomlinson et al. (1995) EMBO J. 14:4628-4638. Amino acid sequences for a repertoire of suitable human germline framework regions can also be obtained from the JOINSOLVER® Germline Databases (e.g., the JOINSOLVER®Kabat databases or the JOINSOLVER® IMGT databases) maintained in part by the U.S. Department of Health and Human Services and the National Institutes of Health. See, e.g., Souto-Carneiro et al. (2004) J Immunol. 172:6790-6802.

Fully human antibodies are antibodies having variable and constant regions (if present) derived from human germline immunoglobulin sequences. Human antibodies can include amino acid residues not encoded by human germline immunoglobulin sequences (e.g., mutations introduced by random or site-specific mutagenesis in vitro or by somatic mutation in vivo). However, the term “human antibody” does not include antibodies in which CDR sequences derived from the germline of another mammalian species, such as a mouse, have been grafted onto human framework sequences (i.e., humanized antibodies). Fully human or human antibodies may be derived from transgenic mice carrying human antibody genes (carrying the variable (V), diversity (D), joining (J), and constant (C) exons) or from human cells. For example, it is possible to produce transgenic animals (e.g., mice) that are capable, upon immunization, of producing a full repertoire of human antibodies in the absence of endogenous immunoglobulin production. (See, e.g., Jakobovits et al. (1993) Proc. Natl. Acad. Sci. USA 90:2551; Jakobovits et al. (1993) Nature 362:255-258; Bruggemann et al. (1993) Year in Immunol. 7:33; and Duchosal et al. (1992) Nature 355:258.) Transgenic mice strains can be engineered to contain gene sequences from unrearranged human immunoglobulin genes. The human sequences may code for both the heavy and light chains of human antibodies and would function correctly in the mice, undergoing rearrangement to provide a wide antibody repertoire similar to that in humans.

The wholly and partially human antibodies described above are less immunogenic than their entirely murine or non-human-derived antibody counterparts. All these molecules (or derivatives thereof) are therefore less likely to evoke an immune or allergic response. Consequently, they are better suited for in vivo administration in humans, especially when repeated or long-term administration is necessary, as may be needed for treatment with the bispecific antibodies described herein (e.g., bispecific antibodies comprising a C5-binding polypeptide described herein and a targeting moiety).

Suitable radioactive labels include, e.g. ³²P, ³³P, ¹⁴C, ¹²⁵I, ¹³¹I, ³⁵S, and ³H. Suitable fluorescent labels include, without limitation, fluorescein, fluorescein isothiocyanate (FITC), green fluorescence protein (GFP), DyLight 488, phycoerythrin (PE), propidium iodide (PI), PerCP, PE-Alexa Fluor® 700, Cy5, allophycocyanin, and Cy7. Luminescent labels include, e.g., any of a variety of luminescent lanthanide (e.g., europium or terbium) chelates. For example, suitable europium chelates include the europium chelate of diethylene triamine pentaacetic acid (DTPA) or tetraazacyclododecane-1,4,7,10-tetraacetic acid (DOTA). Enzymatic labels include, e.g., alkaline phosphatase, CAT, luciferase, and horseradish peroxidase.

Two proteins (e.g., a C5-binding polypeptide and a heterologous moiety) can be cross-linked using any of a number of known chemical cross linkers. Examples of such cross linkers are those which link two amino acid residues via a linkage that includes a “hindered” disulfide bond. In these linkages, a disulfide bond within the cross-linking unit is protected (by hindering groups on either side of the disulfide bond) from reduction by the action, for example, of reduced glutathione or the enzyme disulfide reductase. One suitable reagent, 4-succinimidyloxycarbonyl-α-methyl-α (2-pyridyldithio) toluene (SMPT), forms such a linkage between two proteins utilizing a terminal lysine on one of the proteins and a terminal cysteine on the other. Heterobifunctional reagents that cross-link by a different coupling moiety on each protein can also be used. Other useful cross-linkers include, without limitation, reagents which link two amino groups (e.g., N-5-azido-2-nitrobenzoyloxysuccinimide), two sulfhydryl groups (e.g., 1,4-bis-maleimidobutane), an amino group and a sulfhydryl group (e.g., m-maleimidobenzoyl-N-hydroxysuccinimide ester), an amino group and a carboxyl group (e.g., 4-[p-azidosalicylamido]butylamine), and an amino group and a guanidinium group that is present in the side chain of arginine (e.g., p-azidophenyl glyoxal monohydrate).

In some embodiments, a radioactive label can be directly conjugated to the amino acid backbone of the C5-binding polypeptide. Alternatively, the radioactive label can be included as part of a larger molecule (e.g., ¹²⁵I in meta-[¹²⁵I]iodophenyl-N-hydroxysuccinimide ([¹²⁵I]mIPNHS) which binds to free amino groups to form meta-iodophenyl (mIP) derivatives of relevant proteins (see, e.g., Rogers et al. (1997) Nucl Med 38:1221-1229) or chelate (e.g., to DOTA or DTPA) which is in turn bound to the protein backbone. Methods of conjugating the radioactive labels or larger molecules/chelates containing them to the C5-binding polypeptides described herein are known in the art. Such methods involve incubating the proteins with the radioactive label under conditions (e.g., pH, salt concentration, and/or temperature) that facilitate binding of the radioactive label or chelate to the protein (see, e.g., U.S. Pat. No. 6,001,329).

Methods for conjugating a fluorescent label (sometimes referred to as a “fluorophore”) to a protein (e.g., a C5-binding polypeptide) are known in the art of protein chemistry. For example, fluorophores can be conjugated to free amino groups (e.g., of lysines) or sulfhydryl groups (e.g., cysteines) of proteins using succinimidyl (NHS) ester or tetrafluorophenyl (TFP) ester moieties attached to the fluorophores. In some embodiments, the fluorophores can be conjugated to a heterobifunctional cross-linker moiety such as sulfo-SMCC. Suitable conjugation methods involve incubating a C5-binding polypeptide with the fluorophore under conditions that facilitate binding of the fluorophore to the protein. See, e.g., Welch and Redvanly (2003) “Handbook of Radiopharmaceuticals: Radiochemistry and Applications,” John Wiley and Sons (ISBN 0471495603).

In some embodiments, the C5-binding polypeptides can be modified, e.g., with a moiety that improves the stabilization and/or retention of the antibodies in circulation, e.g., in blood, serum, or other tissues. For example, the C5-binding polypeptide can be PEGylated as described in, e.g., Lee et al. (1999) Bioconjug Chem 10(6): 973-8; Kinstler et al. (2002) Advanced Drug Deliveries Reviews 54:477-485; and Roberts et al. (2002) Advanced Drug Delivery Reviews 54:459-476. The stabilization moiety can improve the stability, or retention of, the polypeptide by at least 1.5 (e.g., at least 2, 5, 10, 15, 20, 25, 30, 40, or 50 or more) fold.

In some embodiments, the C5-binding polypeptides described herein can be glycosylated. In some embodiments, a C5-binding polypeptide described herein can be subjected to enzymatic or chemical treatment, or produced from a cell, such that the antibody has reduced or absent glycosylation. Methods for producing polypeptides with reduced glycosylation are known in the art and described in, e.g., U.S. Pat. No. 6,933,368; Wright et al. (1991) EMBO J. 10(10):2717-2723; and Co et al. (1993) Mol Immunol 30:1361.

### Pharmaceutical Compositions and Formulations

Compositions containing a C5-binding polypeptide described herein can be formulated as a pharmaceutical composition, e.g., for administration to a subject for the treatment or prevention of a complement-associated disorder. The pharmaceutical compositions will generally include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (see e.g., Berge et al. (1977) J Pharm Sci 66:1-19).

The compositions can be formulated according to standard methods. Pharmaceutical formulation is a well-established art, and is further described in, e.g., Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7^{th} Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X). In some embodiments, a composition can be formulated, for example, as a buffered solution at a suitable concentration and suitable for storage at 2-8° C. (e.g., 4° C.). In some embodiments, a composition can be formulated for storage at a temperature below 0° C. (e.g., −20° C. or −80° C.). In some embodiments, the composition can be formulated for storage for up to 2 years (e.g., one month, two months, three months, four months, five months, six months, seven months, eight months, nine months, 10 months, 11 months, 1 year, 1½ years, or 2 years) at 2-8° C. (e.g., 4° C.). Thus, in some embodiments, the compositions described herein are stable in storage for at least 1 year at 2-8° C. (e.g., 4° C.).

The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. For example, compositions containing a C5-binding polypeptide intended for systemic or local delivery can be in the form of injectable or infusible solutions. Accordingly, the compositions can be formulated for administration by a parenteral mode (e.g., intravenous, subcutaneous, intraperitoneal, or intramuscular injection). “Parenteral administration,” “administered parenterally,” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion (see below).

The compositions can be formulated as a solution, microemulsion, dispersion, liposome, or other ordered structure suitable for stable storage at high concentration. Sterile injectable solutions can be prepared by incorporating a C5-binding polypeptide described herein in the required amount in an appropriate solvent with one or a combination of ingredients enumerated above, as required, followed by filtered sterilization. Generally, dispersions are prepared by incorporating a C5-binding polypeptide described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a C5-binding polypeptide described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

The C5-binding polypeptides described herein can also be formulated in immunoliposome compositions. Liposomes containing the antibody can be prepared by methods known in the art such as, e.g., the methods described in Epstein et al. (1985) Proc Natl Acad Sci USA 82:3688; Hwang et al. (1980) Proc Natl Acad Sci USA 77:4030; and U.S. Pat. Nos. 4,485,045 and 4,544,545. Liposomes with enhanced circulation time are disclosed in, e.g., U.S. Pat. No. 5,013,556.

In certain embodiments, a C5-binding polypeptide described herein can be prepared with a carrier that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known in the art. See, e.g., J. R. Robinson (1978) “Sustained and Controlled Release Drug Delivery Systems,” Marcel Dekker, Inc., New York.

In some embodiments, a C5-binding polypeptide described herein can be formulated in a composition suitable for intrapulmonary administration (e.g., for administration via an inhaler or nebulizer) to a mammal such as a human. Methods for preparing such compositions are well known in the art and described in, e.g., U.S. Patent Application Publication No. 20080202513; U.S. Pat. Nos. 7,112,341 and 6,019,968; and PCT Publication Nos. WO 00/061178 and WO 06/122257, the disclosures of each of which are incorporated herein by reference in their entirety. Dry powder inhaler formulations and suitable systems for administration of the formulations are described in, e.g., U.S. Patent Application Publication No. 20070235029, PCT Publication No. WO 00/69887; and U.S. Pat. No. 5,997,848. Additional formulations suitable for intrapulmonary administration (as well as methods for formulating polypeptides) are set forth in, e.g., U.S. Patent Application Publication Nos. 20050271660 and 20090110679.

In some embodiments, a C5-binding polypeptide described herein can be formulated in a composition suitable for delivery to the eye. As used herein, the term “eye” refers to any and all anatomical tissues and structures associated with an eye. The eye has a wall composed of three distinct layers: the outer sclera, the middle choroid layer, and the inner retina. The chamber behind the lens is filled with a gelatinous fluid referred to as the vitreous humor. At the back of the eye is the retina, which detects light. The cornea is an optically transparent tissue, which conveys images to the back of the eye. The cornea includes one pathway for the permeation of drugs into the eye. Other anatomical tissue structures associated with the eye include the lacrimal drainage system, which includes a secretory system, a distributive system and an excretory system. The secretory system comprises secretors that are stimulated by blinking and temperature change due to tear evaporation and reflex secretors that have an efferent parasympathetic nerve supply and secrete tears in response to physical or emotional stimulation. The distributive system includes the eyelids and the tear meniscus around the lid edges of an open eye, which spread tears over the ocular surface by blinking, thus reducing dry areas from developing.

In some embodiments, one or more of the C5-binding polypeptides described herein can be administered locally, for example, by way of topical application or intravitreal injection. For example, in some embodiments, the C5-binding polypeptides can be formulated for administration by way of an eye drop.

The therapeutic preparation for treating the eye can contain one or more C5-binding polypeptides in a concentration from about 0.01 to about 1% by weight, preferably from about 0.05 to about 0.5% in a pharmaceutically acceptable solution, suspension or ointment. The preparation will preferably be in the form of a sterile aqueous solution containing, e.g., additional ingredients such as, but not limited to, preservatives, buffers, tonicity agents, antioxidants and stabilizers, nonionic wetting or clarifying agents, and viscosity-increasing agents. Suitable preservatives for use in such a solution include benzalkonium chloride, benzethonium chloride, chlorobutanol, thimerosal and the like. Suitable buffers include, e.g., boric acid, sodium and potassium bicarbonate, sodium and potassium borates, sodium and potassium carbonate, sodium acetate, and sodium biphosphate, in amounts sufficient to maintain the pH at between about pH 6 and pH 8, and preferably, between about pH 7 and pH 7.5. Suitable tonicity agents are dextran 40, dextran 70, dextrose, glycerin, potassium chloride, propylene glycol, and sodium chloride.

Suitable antioxidants and stabilizers include sodium bisulfite, sodium metabisulfite, sodium thiosulfite, and thiourea. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxymethylpropylcellulose, lanolin, methylcellulose, petrolatum, polyethylene glycol, polyvinyl alcohol, polyvinylpyrrolidone, and carboxymethylcellulose. The preparation can be administered topically to the eye of the subject in need of treatment (e.g., a subject afflicted with AMD) by conventional methods, e.g., in the form of drops, or by bathing the eye in a therapeutic solution, containing one or more C5-binding polypeptides.

In addition, a variety of devices have been developed for introducing drugs into the vitreal cavity of the eye. For example, U.S. patent application publication no. 20020026176 describes a pharmaceutical-containing plug that can be inserted through the sclera such that it projects into the vitreous cavity to deliver the pharmaceutical agent into the vitreous cavity. In another example, U.S. Pat. No. 5,443,505 describes an implantable device for introduction into a suprachoroidal space or an avascular region for sustained release of drug into the interior of the eye. U.S. Pat. Nos. 5,773,019 and 6,001,386 each disclose an implantable drug delivery device attachable to the scleral surface of an eye. The device comprises an inner core containing an effective amount of a low solubility agent covered by a non-bioerodible polymer that is permeable to the low solubility agent. During operation, the low solubility agent permeates the bioerodible polymer cover for sustained release out of the device. Additional methods and devices (e.g., a transscleral patch and delivery via contact lenses) for delivery of a therapeutic agent to the eye are described in, e.g., Ambati and Adamis (2002) Prog Retin Eye Res 21(2):145-151; Ranta and Urtti (2006) Adv Drug Delivery Rev 58(11):1164-1181; Barocas and Balachandran (2008) Expert Opin Drug Delivery 5(1):1-10(10); Gulsen and Chauhan (2004) Invest Ophthalmol V is Sci 45:2342-2347; Kim et al. (2007) Ophthalmic Res 39:244-254; and PCT publication no. WO 04/073551, the disclosures of which are incorporated herein by reference in their entirety.

As described above, the C5-binding polypeptides described herein can be formulated as relatively high concentrations in aqueous pharmaceutical solutions. For example, the C5-binding polypeptides can be formulated in solution at a concentration of between about 10 mg/mL to 100 mg/mL (e.g., between about 9 mg/mL and 90 mg/mL; between about 9 mg/mL and 50 mg/mL; between about 10 mg/mL and 50 mg/mL; between about 15 mg/mL and 50 mg/mL; between about 15 mg/mL and 110 mg/mL; between about 15 mg/mL and 100 mg/mL; between about 20 mg/mL and 100 mg/mL; between about 20 mg/mL and 80 mg/mL; between about 25 mg/mL and 100 mg/mL; between about 25 mg/mL and 85 mg/mL; between about 20 mg/mL and 50 mg/mL; between about 25 mg/mL and 50 mg/mL; between about 30 mg/mL and 100 mg/mL; between about 30 mg/mL and 50 mg/mL; between about 40 mg/mL and 100 mg/mL; between about 50 mg/mL and 100 mg/mL; or between about 20 mg/mL and 50 mg/mL). In some embodiments, the polypeptide is present in the solution at greater than (or at least or equal to) 5 (e.g., greater than, at least, or equal to: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, or even 150) mg/mL. In some embodiments, a C5-binding polypeptide can be formulated at a concentration of greater than 2 (e.g., greater than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 or more) mg/mL, but less than 55 (e.g., less than 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or less than 5) mg/mL. Thus, in some embodiments, a C5-binding polypeptide can be formulated in an aqueous solution at a concentration of greater than 5 mg/mL and less than 50 mg/mL. In some embodiments, a C5-binding polypeptide can be formulated in an aqueous solution at a concentration of about 50 mg/mL. Methods for formulating a protein in an aqueous solution are known in the art and are described in, e.g., U.S. Pat. No. 7,390,786; McNally and Hastedt (2007), “Protein Formulation and Delivery,” Second Edition, Drugs and the Pharmaceutical Sciences, Volume 175, CRC Press; and Banga (1995), “Therapeutic peptides and proteins: formulation, processing, and delivery systems,” CRC Press. In some embodiments, the aqueous solution has a neutral pH, e.g., a pH between, e.g., 6.5 and 8 (e.g., between and inclusive of 7 and 8). In some embodiments, the aqueous solution has a pH of about 6.6, 6.7, 6.8, 6.9, 7, 7.1, 7.2, 7.3, 7.4, 7.5, 7.6, 7.7, 7.8, 7.9, or 8.0. In some embodiments, the aqueous solution has a pH of greater than (or equal to) 6 (e.g., greater than or equal to 6.1, 6.2, 6.3, 6.4, 6.5, 6.6, 6.7, 6.8, 6.9, 7, 7.1, 7.2, 7.3, 7.4, 7.5, 7.6, 7.7, 7.8, or 7.9), but less than pH 8.

Nucleic acids encoding a C5-binding polypeptide can be incorporated into a gene construct to be used as a part of a gene therapy protocol to deliver nucleic acids that can be used to express and produce agents within cells (see below). Expression constructs of such components may be administered in any therapeutically effective carrier, e.g. any formulation or composition capable of effectively delivering the component gene to cells in vivo. Approaches include insertion of the subject gene in viral vectors including recombinant retroviruses, adenovirus, adeno-associated virus, lentivirus, and herpes simplex virus-1 (HSV-1), or recombinant bacterial or eukaryotic plasmids. Viral vectors can transfect cells directly; plasmid DNA can be delivered with the help of, for example, cationic liposomes (lipofectin) or derivatized (e.g., antibody conjugated), polylysine conjugates, gramicidin S, artificial viral envelopes or other such intracellular carriers, as well as direct injection of the gene construct or CaPO₄ precipitation (see, e.g., WO04/060407) carried out in vivo. (See also, “Ex vivo Approaches,” below.) Examples of suitable retroviruses include pLJ, pZIP, pWE and pEM which are known to those skilled in the art (see, e.g., Eglitis et al. (1985) Science 230:1395-1398; Danos and Mulligan (1988) Proc Natl Acad Sci USA 85:6460-6464; Wilson et al. (1988) Proc Natl Acad Sci USA 85:3014-3018; Armentano et al. (1990) Proc. Natl. Acad. Sci. USA 87:6141-6145; Huber et al. (1991) Proc Natl Acad Sci USA 88:8039-8043; Ferry et al. (1991) Proc Natl Acad Sci USA 88:8377-8381; Chowdhury et al. (1991) Science 254:1802-1805; van Beusechem et al. (1992) Proc Natl Acad Sci USA 89:7640-7644; Kay et al. (1992) Human Gene Therapy 3:641-647; Dai et al. (1992) Proc Natl Acad Sci USA 89:10892-10895; Hwu et al. (1993) J Immunol 150:4104-4115; U.S. Pat. Nos. 4,868,116 and 4,980,286; PCT Publication Nos. WO89/07136, WO89/02468, WO89/05345, and WO92/07573). Another viral gene delivery system utilizes adenovirus-derived vectors (see, e.g., Berkner et al. (1988) BioTechniques 6:616; Rosenfeld et al. (1991) Science 252:431-434; and Rosenfeld et al. (1992) Cell 68:143-155). Suitable adenoviral vectors derived from the adenovirus strain Ad type 5 d1324 or other strains of adenovirus (e.g., Ad2, Ad3, Ad7, etc.) are known to those skilled in the art. Yet another viral vector system useful for delivery of the subject gene is the adeno-associated virus (AAV). See, e.g., Flotte et al. (1992) Am J Respir Cell Mol Biol 7:349-356; Samulski et al. (1989) J Virol 63:3822-3828; and McLaughlin et al. (1989) J Virol 62:1963-1973.

In some embodiments, a C5-binding polypeptide described herein can be formulated with one or more additional active agents useful for treating or preventing a complement-associated disorder (e.g., an AP-associated disorder or a CP-associated disorder) in a subject. Additional agents for treating a complement-associated disorder in a subject will vary depending on the particular disorder being treated, but can include, without limitation, an antihypertensive (e.g., an angiotensin-converting enzyme inhibitor) [for use in treating, e.g., HELLP syndrome], an anticoagulant, a corticosteroid (e.g., prednisone), or an immunosuppressive agent (e.g., vincristine or cyclosporine A). Examples of anticoagulants include, e.g., warfarin (Coumadin), aspirin, heparin, phenindione, fondaparinux, idraparinux, and thrombin inhibitors (e.g., argatroban, lepirudin, bivalirudin, or dabigatran). A C5-binding polypeptide described herein can also be formulated with a fibrinolytic agent (e.g., ancrod, ε-aminocaproic acid, antiplasmin-a₁, prostacyclin, and defibrotide) for the treatment of a complement-associated disorder. In some embodiments, a C5-binding polypeptide can be formulated with a lipid-lowering agent such as an inhibitor of hydroxymethylglutaryl CoA reductase. In some embodiments, a C5-binding polypeptide can be formulated with, or for use with, an anti-CD20 agent such as rituximab (Rituxan™; Biogen Idec, Cambridge, Mass.). In some embodiments, e.g., for the treatment of RA, the C5-binding polypeptide can be formulated with one or both of infliximab (Remicade®; Centocor, Inc.) and methotrexate (Rheumatrex®, Trexall®). In some embodiments, a C5-binding polypeptide described herein can be formulated with a non-steroidal anti-inflammatory drug (NSAID). Many different NSAIDS are available, some over the counter including ibuprofen (Advil®, Motrin®, Nuprin®) and naproxen (Alleve®) and many others are available by prescription including meloxicam (Mobic®), etodolac (Lodine®), nabumetone (Relafen®), sulindac (Clinoril®), tolementin (Tolectin®), choline magnesium salicylate (Trilasate®), diclofenac (Cataflam®, Voltaren®, Arthrotec®), Diflusinal (Dolobid®), indomethicin (Indocin®), Ketoprofen (Orudis®, Oruvail®), Oxaprozin (Daypro®), and piroxicam (Feldene®). In some embodiments a C5-binding polypeptide can be formulated for use with an anti-hypertensive, an anti-seizure agent (e.g., magnesium sulfate), or an anti-thrombotic agent. Anti-hypertensives include, e.g., labetalol, hydralazine, nifedipine, calcium channel antagonists, nitroglycerin, or sodium nitroprussiate. (See, e.g., Mihu et al. (2007) J Gastrointestin Liver Dis 16(4):419-424.) Anti-thrombotic agents include, e.g., heparin, antithrombin, prostacyclin, or low dose aspirin.

In some embodiments, a C5-binding polypeptide described herein can be formulated for administration (e.g., intrapulmonary administration) with at least one additional active agent for treating a pulmonary disorder. The at least one active agent can be, e.g., an anti-IgE antibody (e.g., omalizumab), an anti-IL-4 antibody or an anti-IL-5 antibody, an anti-IgE inhibitor (e.g., montelukast sodium), a sympathomimetic (e.g., albuterol), an antibiotic (e.g., tobramycin), a deoxyribonuclease (e.g., pulmozyme), an anticholinergic drug (e.g., ipratropium bromide), a corticosteroid (e.g., dexamethasone), a β-adrenoreceptor agonist, a leukotriene inhibitor (e.g., zileuton), a 5-lipoxygenase inhibitor, a PDE inhibitor, a CD23 antagonist, an IL-13 antagonist, a cytokine release inhibitor, a histamine H1 receptor antagonist, an anti-histamine, an anti-inflammatory agent (e.g., cromolyn sodium), or a histamine release inhibitor.

In some embodiments, a C5-binding polypeptide described herein can be formulated for administration with one or more additional therapeutic agents for use in treating a complement-associated disorder of the eye. Such additional therapeutic agents can be, e.g., bevacizumab or the Fab fragment of bevacizumab or ranibizumab, both sold by Roche Pharmaceuticals, Inc., and pegaptanib sodium (Mucogen®; Pfizer, Inc.). Such a kit can also, optionally, include instructions for administering the C5-binding polypeptide to a subject.

In some embodiments, a C5-binding polypeptide described herein can be formulated for administration to a subject along with intravenous gamma globulin therapy (IVIG), plasmapheresis, plasma replacement, or plasma exchange. In some embodiments, a C5-binding polypeptide can be formulated for use before, during, or after, a kidney transplant.

When a C5-binding polypeptide is to be used in combination with a second active agent, the agents can be formulated separately or together. For example, the respective pharmaceutical compositions can be mixed, e.g., just prior to administration, and administered together or can be administered separately, e.g., at the same or different times (see below).

As described above, a composition can be formulated such that it includes a therapeutically effective amount of a C5-binding polypeptide described herein. In some embodiments, a composition can be formulated to include a sub-therapeutic amount of a C5-binding polypeptide and a sub-therapeutic amount of one or more additional active agents such that the components in total are therapeutically effective for treating or preventing a complement-associated disorder (e.g., an alternative complement pathway-associated complement disorder or a classical complement pathway-associated disorder) in a subject. Methods for determining a therapeutically effective dose of an agent such as a therapeutic antibody are known in the art and described herein.

### Applications

The C5-binding polypeptides, conjugates thereof, and compositions of any of the foregoing can be used in a number of diagnostic and therapeutic applications. For example, detectably-labeled C5-binding polypeptides can be used in assays to detect the presence or amount of C5 present in a biological sample. Suitable methods for using the antibodies in diagnostic assays are known in the art and include, without limitation, ELISA, fluorescence resonance energy transfer applications, Western blot, and dot blot techniques. See, e.g., Sambrook et al., supra and Ausubel et al., supra.

In some embodiments, the C5-binding polypeptides described herein can be used as positive controls in assays designed to identify additional novel compounds for treating complement-mediated disorders. For example, a C5-binding polypeptide that inhibits formation of terminal complement and/or C5a production can be used as a positive control in an assay to identify additional compounds (e.g., small molecules, aptamers, or antibodies) that reduce or abrogate C5a production or formation of MAC.

In some embodiments, mouse C5-binding polypeptides described herein can be used as a surrogate antibody in mouse models of human disease. This can be especially useful where a human C5-binding polypeptide (e.g., a single chain anti-C5 antibody) does not cross-react with mouse C5 and/or is likely to cause an anti-human antibody response in a mouse to which the humanized antibody is administered. Accordingly, a researcher wishing to study the effect of a C5-binding polypeptide in treating a disease (e.g., AMD, asthma, or RA) can use a mouse C5-binding polypeptide described herein in an appropriate mouse model of the disease. If the researcher can establish efficacy in the mouse model of disease using the mouse C5-binding polypeptide, the results may establish proof-of-concept for use of a human C5-binding polypeptide in treating the disease in humans.

The C5-binding polypeptides described herein can also be used in therapeutic methods as elaborated on below.

### Methods for Treatment

The above-described compositions (e.g., any of the C5-binding polypeptides described herein or pharmaceutical compositions thereof) are useful in, inter alia, methods for treating or preventing a variety of complement-associated disorders (e.g., AP-associated disorders or CP-associated disorders) in a subject. The compositions can be administered to a subject, e.g., a human subject, using a variety of methods that depend, in part, on the route of administration. The route can be, e.g., intravenous injection or infusion (IV), subcutaneous injection (SC), intraperitoneal (IP) injection, intrapulmonary injection, intraocular injection, intraarticular injection, or intramuscular injection (IM).

In some embodiments, a C5-binding polypeptide is therapeutically delivered to a subject by way of local administration. As used herein, “local administration” or “local delivery,” refers to delivery that does not rely upon transport of the composition or agent to its intended target tissue or site via the vascular system. For example, the composition may be delivered by injection or implantation of the composition or agent or by injection or implantation of a device containing the composition or agent. Following local administration in the vicinity of a target tissue or site, the composition or agent, or one or more components thereof, may diffuse to the intended target tissue or site.

In some embodiments, a C5-binding polypeptide can be locally administered to a joint (e.g., an articulated joint). For example, in embodiments where the complement-associated disorder is arthritis, the polypeptide can be administered directly to a joint (e.g., into a joint space) or in the vicinity of a joint. Examples of intraarticular joints to which a C5-binding polypeptide can be locally administered include, e.g., the hip, knee, elbow, wrist, sternoclavicular, temperomandibular, carpal, tarsal, ankle, and any other joint subject to arthritic conditions. A C5-binding polypeptide can also be administered to bursa such as, e.g., acromial, bicipitoradial, cubitoradial, deltoid, infrapatellar, ischial, and any other bursa known in the art of medicine.

In some embodiments, a C5-binding polypeptide can be locally administered to the eye, e.g., to treat patients afflicted with a complement-associated disorder of the eye such as wet or dry AMD. As used herein, the term “eye” refers to any and all anatomical tissues and structures associated with an eye. The eye has a wall composed of three distinct layers: the outer sclera, the middle choroid layer, and the inner retina. The chamber behind the lens is filled with a gelatinous fluid referred to as the vitreous humor. At the back of the eye is the retina, which detects light. The cornea is an optically transparent tissue, which conveys images to the back of the eye. The cornea includes one pathway for the permeation of drugs into the eye. Other anatomical tissue structures associated with the eye include the lacrimal drainage system, which includes a secretory system, a distributive system and an excretory system. The secretory system comprises secretors that are stimulated by blinking and temperature change due to tear evaporation and reflex secretors that have an efferent parasympathetic nerve supply and secrete tears in response to physical or emotional stimulation. The distributive system includes the eyelids and the tear meniscus around the lid edges of an open eye, which spread tears over the ocular surface by blinking, thus reducing dry areas from developing.

In some embodiments, a C5-binding polypeptide is administered to the posterior chamber of the eye. In some embodiments, a C5-binding polypeptide is administered intravitreally. In some embodiments, a C5-binding polypeptide is administered trans-sclerally.

In some embodiments, e.g., in embodiments for treatment or prevention of a complement-associated pulmonary disorder such as COPD or asthma, a C5-binding polypeptide described herein can also be administered to a subject by way of the lung. Pulmonary drug delivery may be achieved by inhalation, and administration by inhalation herein may be oral and/or nasal. Examples of pharmaceutical devices for pulmonary delivery include metered dose inhalers, dry powder inhalers (DPIs), and nebulizers. For example, a C5-binding polypeptide can be administered to the lungs of a subject by way of a dry powder inhaler. These inhalers are propellant-free devices that deliver dispersible and stable dry powder formulations to the lungs. Dry powder inhalers are well known in the art of medicine and include, without limitation: the TurboHaler® (AstraZeneca; London, England) the AIR® inhaler (Alkermes®; Cambridge, Mass.); Rotahaler® (GlaxoSmithKline; London, England); and Eclipse™ (Sanofi-Aventis; Paris, France). See also, e.g., PCT Publication Nos. WO 04/026380, WO 04/024156, and WO 01/78693. DPI devices have been used for pulmonary administration of polypeptides such as insulin and growth hormone. In some embodiments, a C5-binding polypeptide described herein can be intrapulmonarily administered by way of a metered dose inhaler. These inhalers rely on a propellant to deliver a discrete dose of a compound to the lungs. Examples of compounds administered by metered dose inhalers include, e.g., Astovent®(Boehringer-Ingelheim; Ridgefield, Conn.) and Flovent® (GlaxoSmithKline). See also, e.g., U.S. Pat. Nos. 6,170,717; 5,447,150; and 6,095,141.

In some embodiments, a C5-binding polypeptide can be administered to the lungs of a subject by way of a nebulizer. Nebulizers use compressed air to deliver a compound as a liquefied aerosol or mist. A nebulizer can be, e.g., a jet nebulizer (e.g., air or liquid-jet nebulizers) or an ultrasonic nebulizer. Additional devices and intrapulmonary administration methods are set forth in, e.g., U.S. Patent Application Publication Nos. 20050271660 and 20090110679, the disclosures of each of which are incorporated herein by reference in their entirety.

In some embodiments, a C5-binding polypeptide described herein is administered by way of intrapulmonary administration to a subject in need thereof. For example, one or more of the C5-binding polypeptides can be delivered by way of a nebulizer or an inhaler to a subject (e.g., a human) afflicted with a complement-associated pulmonary disorder such as asthma or COPD.

It is understood that in some embodiments one or more of the C5-binding polypeptides described herein can be administered systemically for use in treating, e.g., RA, wet or dry AMD, asthma, and/or COPD.

A suitable dose of a C5-binding polypeptide described herein, which dose is capable of treating or preventing a complement-associated disorder in a subject, can depend on a variety of factors including, e.g., the age, sex, and weight of a subject to be treated and the particular inhibitor compound used. For example, a different dose of a C5-binding polypeptide may be required to treat an elderly subject with RA as compared to the dose of a C5-binding polypeptide that is required to treat a younger subject. Other factors affecting the dose administered to the subject include, e.g., the type or severity of the complement-associated disorder. For example, a subject having RA may require administration of a different dosage of a C5-binding polypeptide than a subject with AMD. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject will depend upon the judgment of the treating medical practitioner (e.g., doctor or nurse).

An antibody described herein can be administered as a fixed dose, or in a milligram per kilogram (mg/kg) dose. In some embodiments, the dose can also be chosen to reduce or avoid production of antibodies or other host immune responses against one or more of the active antibodies in the composition. While in no way intended to be limiting, exemplary dosages of an antibody include, e.g., 1-100 μg/kg, 0.5-50 μg/kg, 0.1-100 μg/kg, 0.5-25 μg/kg, 1-20 μg/kg, and 1-10 μg/kg, 1-100 mg/kg, 0.5-50 mg/kg, 0.1-100 mg/kg, 0.5-25 mg/kg, 1-20 mg/kg, and 1-10 mg/kg. Exemplary dosages of an antibody described herein include, without limitation, 0.1 μg/kg, 0.5 μg/kg, 1.0 μg/kg, 2.0 μg/kg, 4 μg/kg, and 8 μg/kg, 0.1 mg/kg, 0.5 mg/kg, 1.0 mg/kg, 2.0 mg/kg, 4 mg/kg, and 8 mg/kg.

A pharmaceutical composition can include a therapeutically effective amount of an antibody described herein. Such effective amounts can be readily determined by one of ordinary skill in the art based, in part, on the effect of the administered antibody, or the combinatorial effect of the antibody and one or more additional active agents, if more than one agent is used. A therapeutically effective amount of an antibody described herein can also vary according to factors such as the disease state, age, sex, and weight of the individual, and the ability of the antibody (and one or more additional active agents) to elicit a desired response in the individual, e.g., amelioration of at least one condition parameter, e.g., amelioration of at least one symptom of the complement-associated disorder. For example, a therapeutically effective amount of a C5-binding polypeptide can inhibit (lessen the severity of or eliminate the occurrence of) and/or prevent a particular disorder, and/or any one of the symptoms of the particular disorder known in the art or described herein. A therapeutically effective amount is also one in which any toxic or detrimental effects of the composition are outweighed by the therapeutically beneficial effects.

Suitable human doses of any of the C5-binding polypeptides described herein can further be evaluated in, e.g., Phase I dose escalation studies. See, e.g., van Gurp et al. (2008) Am J Transplantation 8(8):1711-1718; Hanouska et al. (2007) Clin Cancer Res 13(2, part 1):523-531; and Hetherington et al. (2006) Antimicrobial Agents and Chemotherapy 50(10): 3499-3500.

While in no way intended to be limiting, exemplary methods of administration for a single chain antibody such as a single chain anti-C5 antibody (that inhibits cleavage of C5) are described in, e.g., Granger et al. (2003) Circulation 108:1184; Haverich et al. (2006) Ann Thorac Surg 82:486-492; and Testa et al. (2008) J Thorac Cardiovasc Surg 136(4):884-893.

The terms “therapeutically effective amount” or “therapeutically effective dose,” or similar terms used herein are intended to mean an amount of an agent that will elicit the desired biological or medical response (e.g., an improvement in one or more symptoms of a complement-associated disorder). In some embodiments, a composition described herein contains a therapeutically effective amount of a C5-binding polypeptide. In some embodiments, the composition contains any of the C5-binding polypeptides described herein and one or more (e.g., one, two, three, four, five, six, seven, eight, nine, 10, or 11 or more) additional therapeutic agents such that the composition as a whole is therapeutically effective. For example, a composition can contain a C5-binding polypeptide described herein and an immunosuppressive agent, wherein the polypeptide and agent are each at a concentration that when combined are therapeutically effective for treating or preventing a complement-associated disorder in a subject.

Toxicity and therapeutic efficacy of such compositions can be determined by known pharmaceutical procedures in cell cultures or experimental animals (e.g., animal models of any of the complement-associated disorders described herein). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. A C5-binding polypeptide that exhibits a high therapeutic index is preferred. While compositions that exhibit toxic side effects may be used, care should be taken to design a delivery system that targets such compounds to the site of affected tissue and to minimize potential damage to normal cells and, thereby, reduce side effects.

The data obtained from the cell culture assays and animal studies can be used in formulating a range of dosage for use in humans. The dosage of such antibodies lies generally within a range of circulating concentrations of the C5-binding polypeptides that include the ED₅₀ with little or no toxicity. The dosage may vary within this range depending upon the dosage form employed and the route of administration utilized. For a C5-binding polypeptide used as described herein (e.g., for treating or preventing a complement-associated disorder), the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography or by ELISA.

In some embodiments, the methods can be performed in conjunction with other therapies for complement-associated disorders. For example, the composition can be administered to a subject at the same time, prior to, or after, plasmapheresis, IVIG therapy, plasma replacement, or plasma exchange. See, e.g., Appel et al. (2005) J Am Soc Nephrol 16:1392-1404. In some embodiments, a C5-binding polypeptide described herein is not administered in conjunction with IVIG. In some embodiments, the composition can be administered to a subject at the same time, prior to, or after, a kidney transplant.

A “subject,” as used herein, can be any mammal. For example, a subject can be a human, a non-human primate (e.g., monkey, baboon, or chimpanzee), a horse, a cow, a pig, a sheep, a goat, a dog, a cat, a rabbit, a guinea pig, a gerbil, a hamster, a rat, or a mouse. In some embodiments, the subject is an infant (e.g., a human infant).

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment (such as treatment with a composition comprising a C5-binding polypeptide).

As described above, the C5-binding polypeptides described herein can be used to treat a variety of complement-associated disorders such as, e.g., AP-associated disorders and/or CP-associated disorders. Such disorders include, without limitation, rheumatoid arthritis (RA); antiphospholipid antibody syndrome; lupus nephritis; pulmonary disorders; ischemia-reperfusion injury; atypical hemolytic uremic syndrome (aHUS); typical or infectious hemolytic uremic syndrome (tHUS); dense deposit disease (DDD); paroxysmal nocturnal hemoglobinuria (PNH); neuromyelitis optica (NMO); multifocal motor neuropathy (MMN); multiple sclerosis (MS); macular degeneration (e.g., age-related macular degeneration (AMD)); hemolysis, elevated liver enzymes, and low platelets (HELLP) syndrome; thrombotic thrombocytopenic purpura (TTP); spontaneous fetal loss; Pauci-immune vasculitis; epidermolysis bullosa; recurrent fetal loss; and traumatic brain injury. (See, e.g., Holers (2008) Immunological Reviews 223:300-316 and Holers and Thurman (2004) Molecular Immunology 41:147-152.) In some embodiments, the complement-associated disorder is a complement-associated vascular disorder such as, but not limited to, a cardiovascular disorder, myocarditis, a cerebrovascular disorder, a peripheral (e.g., musculoskeletal) vascular disorder, a renovascular disorder, a mesenteric/enteric vascular disorder, revascularization to transplants and/or replants, vasculitis, Henoch-Schönlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease (arteritis), venous gas embolus (VGE), and restenosis following stent placement, rotational atherectomy, and percutaneous transluminal coronary angioplasty (PTCA). (See, e.g., U.S. patent application publication no. 20070172483.) Additional complement-associated disorders include, without limitation, MG, CAD, dermatomyositis, Graves' disease, atherosclerosis, Alzheimer's disease, Guillain-Barré Syndrome, Degos' disease, graft rejection (e.g., transplant rejection), systemic inflammatory response sepsis, septic shock, spinal cord injury, glomerulonephritis, Hashimoto's thyroiditis, type I diabetes, psoriasis, pemphigus, autoimmune hemolytic anemia (AIHA), idiopathic thrombocytopenic purpura (ITP), Goodpasture syndrome, antiphospholipid syndrome (APS), and catastrophic APS (CAPS). Pulmonary disorders include, e.g., chronic obstructive pulmonary disorder (COPD), asthma, pulmonary fibrosis, bronchitis, emphysema, bronchiolitis obliterans, and sarcoidosis. Additional pulmonary disorders that can be treated or prevented using the compositions and methods described herein are set forth in, e.g., U.S. Patent Application Publication No. 20050271660. In some embodiments, the C5-binding polypeptides described herein can be used in methods for treating thrombotic microangiopathy (TMA), e.g., TMA associated with a complement-associated disorder such as any of the complement-associated disorders described herein.

As used herein, a subject “at risk for developing a complement-associated disorder” (e.g., an AP-associated disorder or a CP-associated disorder) is a subject having one or more (e.g., two, three, four, five, six, seven, or eight or more) risk factors for developing the disorder. Risk factors will vary depending on the particular complement-associated disorder, but are well known in the art of medicine. For example, risk factors for developing DDD include, e.g., a predisposition to develop the condition, i.e., a family history of the condition or a genetic predisposition to develop the condition such as, e.g., one or more mutations in the gene encoding complement factor H(CFH), complement factor H-related 5 (CFHR5), and/or complement component C3 (C3). Such DDD-associated mutations as well methods for determining whether a subject carries one or more of the mutations are known in the art and described in, e.g., Licht et al. (2006) Kidney Int 70:42-50; Zipfel et al. (2006) “The role of complement in membranoproliferative glomerulonephritis,” In: Complement and Kidney Disease, Springer, Berlin, pages 199-221; Ault et al. (1997) Biol Chem 272:25168-75; Abrera-Abeleda et al. (2006) J Med Genet. 43:582-589; Poznansky et al. (1989) J Immunol 143:1254-1258; Jansen et al. (1998) Kidney Int 53:331-349; and Hegasy et al. (2002) Am J Pathol 161:2027-2034. Thus, a human at risk for developing DDD can be, e.g., one who has one or more DDD-associated mutations in the gene encoding CFH or one with a family history of developing the disease.

Risk factors for TTP are well known in the art of medicine and include, e.g., a predisposition to develop the condition, i.e., a family history of the condition or a genetic predisposition to develop the condition such as, e.g., one or more mutations in the ADAMTS13 gene. ADAMTS13 mutations associated with TTP are reviewed in detail in, e.g., Levy et al. (2001) Nature 413:488-494; Kokame et al. (2004) Semin Hematol 41:34-40; Licht et al. (2004) Kidney Int 66:955-958; and Noris et al. (2005) J Am Soc Nephrol 16:1177-1183. Risk factors for TTP also include those conditions or agents that are known to precipitate TTP, or TTP recurrence, such as, but not limited to, cancer, bacterial infections (e.g., Bartonella sp. infections), viral infections (e.g., HIV and Kaposi's sarcoma virus), pregnancy, or surgery. See, e.g., Avery et al. (1998) Am J Hematol 58:148-149 and Tsai, supra. TTP, or recurrence of TTP, has also been associated with the use of certain therapeutic agents (drugs) including, e.g., ticlopidine, FK506, corticosteroids, tamoxifen, or cyclosporin A (see, e.g., Gordon et al. (1997) Sem Hematol 34(2):140-147). Hereinafter, such manifestations of TTP may be, where appropriate, referred to as, e.g., “infection-associated TTP,” “pregnancy-associated TTP,” or “drug-associated TTP.” Thus, a human at risk for developing TTP can be, e.g., one who has one or more TTP-associated mutations in the ADAMTS13 gene. A human at risk for developing a recurrent form of TTP can be one, e.g., who has had TTP and has an infection, is pregnant, or is undergoing surgery.

Risk factors for aHUS are well known in the art of medicine and include, e.g., a predisposition to develop the condition, i.e., a family history of the condition or a genetic predisposition to develop the condition such as, e.g., one or more mutations in complement Factor H(CFH), membrane cofactor protein (MCP; CD46), C4b-binding protein, complement factor B (CFB), or complement factor I (CFI). (See, e.g., Warwicker et al. (1998) Kidney Int 53:836-844; Richards et al. (2001) Am J Hum Genet. 68:485-490; Caprioli et al. (2001) Am Soc Nephrol 12:297-307; Neuman et al. (2003) J Med Genet. 40:676-681; Richards et al. (2006) Proc Natl Acad Sci USA 100:12966-12971; Fremeaux-Bacchi et al. (2005) J Am Soc Nephrol 17:2017-2025; Esparza-Gordillo et al. (2005) Hum Mol Genet. 14:703-712; Goicoechea de Jorge et al. (2007) Proc Natl Acad Sci USA 104(1):240-245; Blom et al. (2008) J Immunol 180(9):6385-91; and Fremeaux-Bacchi et al. (2004) J Med Genet. 41:e84). (See also Kavanagh et al. (2006) supra.) Risk factors also include, e.g., infection with Streptococcus pneumoniae, pregnancy, cancer, exposure to anti-cancer agents (e.g., quinine, mitomycin C, cisplatin, or bleomycin), exposure to immunotherapeutic agents (e.g., cyclosporine, OKT3, or interferon), exposure to anti-platelet agents (e.g., ticlopidine or clopidogrel), HIV infection, transplantation, autoimmune disease, and combined methylmalonic aciduria and homocystinuria (cb1C). See, e.g., Constantinescu et al. (2004) Am J Kidney Dis 43:976-982; George (2003) Curr Opin Hematol 10:339-344; Gottschall et al. (1994) Am J Hematol 47:283-289; Valavaara et al. (1985) Cancer 55:47-50; Miralbell et al. (1996) J Clin Oncol 14:579-585; Dragon-Durey et al. (2005) J Am Soc Nephrol 16:555-63; and Becker et al. (2004) Clin Infect Dis 39:S267-S275.

Risk factors for HELLP are well known in the art of medicine and include, e.g., multiparous pregnancy, maternal age over 25 years, Caucasian race, the occurrence of preeclampsia or HELLP in a previous pregnancy, and a history of poor pregnancy outcome. (See, e.g., Sahin et al. (2001) Nagoya Med J44(3):145-152; Sullivan et al. (1994) Am J Obstet Gynecol 171:940-943; and Padden et al. (1999) Am Fam Physician 60(3):829-836.) For example, a pregnant, Caucasian woman who developed preeclampsia during a first pregnancy can be one at risk for developing HELLP syndrome during, or following, a second pregnancy.

Risk factors for CAD are well known in the art of medicine and include, e.g., conditions or agents that are known to precipitate CAD, or CAD recurrence, such as, but not limited to, neoplasms or infections (e.g., bacterial and viral infections). Conditions known to be associated with the development of CAD include, e.g., HIV infection (and AIDS), hepatitis C infection, Mycoplasma pneumonia infection, Epstein-Barr virus (EBV) infection, cytomegalovirus (CMV) infection, rubella, or infectious mononucleosis. Neoplasms associated with CAD include, without limitation, non-Hodgkin's lymphoma. Hereinafter, such manifestations of CAD may be, where appropriate, referred to as, e.g., “infection-associated CAD” or “neoplasm-associated CAD.” Thus, a human at risk for developing CAD can be, e.g., one who has an HIV infection, rubella, or a lymphoma. See also, e.g., Gertz (2006) Hematology 1:19-23; Horwitz et al. (1977) Blood 50:195-202; Finland and Barnes (1958) AMA Arch Intern Med 191:462-466; Wang et al. (2004) Acta Paediatr Taiwan 45:293-295; Michaux et al. (1998) Ann Hematol 76:201-204; and Chang et al. (2004) Cancer Genet Cytogenet 152:66-69.

Risk factors for myasthenia gravis (MG) are well known in the art of medicine and include, e.g., a predisposition to develop the condition, i.e., a family history of the condition or a genetic predisposition to develop the condition such as familial MG. For example, some HLA types are associated with an increased risk for developing MG. Risk factors for MG include the ingestion or exposure to certain MG-inducing drugs such as, but not limited to, D-penicillamine. See, e.g., Drosos et al. (1993) Clin Exp Rheumatol 11(4):387-91 and Kaeser et al. (1984) Acta Neurol Scand Suppl 100:39-47. As MG can be episodic, a subject who has previously experienced one or more symptoms of having MG can be at risk for relapse. Thus, a human at risk for developing MG can be, e.g., one who has a family history of MG and/or one who has ingested or been administered an MG-inducing drug such as D-penicillamine.

As used herein, a subject “at risk for developing CAPS” is a subject having one or more (e.g., two, three, four, five, six, seven, or eight or more) risk factors for developing the disorder. Approximately 60% of the incidences of CAPS are preceded by a precipitating event such as an infection. Thus, risk factors for CAPS include those conditions known to precipitate CAPS such as, but not limited to, certain cancers (e.g., gastric cancer, ovarian cancer, lymphoma, leukemia, endometrial cancer, adenocarcinoma, and lung cancer), pregnancy, puerperium, transplantation, primary APS, rheumatoid arthritis (RA), systemic lupus erythematosus (SLE), surgery (e.g., eye surgery), and certain infections. Infections include, e.g., parvovirus B19 infection and hepatitis C infection. Hereinafter, such manifestations of CAPS may be referred to as, e.g., “cancer-associated CAPS,” “transplantation-associated CAPS,” “RA-associated CAPS,” “infection-associated CAPS,” or “SLE-associated CAPS.” See, e.g., Soltesz et al. (2000) Haematologia (Budep) 30(4):303-311; Ideguchi et al. (2007) Lupus 16(1):59-64; Manner et al. (2008) Am J Med Sci 335(5):394-7; Miesbach et al. (2006) Autoimmune Rev 6(2):94-7; Gómez-Puerta et al. (2006) Autoimmune Rev 6(2):85-8; Gomez-Puerta et al. (2006) Semin Arthritis Rheum 35(5):322-32; Kasamon et al. (2005) Haematologia 90(3):50-53; Atherson et al. (1998) Medicine 77(3):195-207; and Canpolat et al. (2008) Clin Pediatr 47(6):593-7. Thus, a human at risk for developing CAPS can be, e.g., one who has primary CAPS and/or a cancer that is known to be associated with CAPS.

From the above it will be clear that subjects “at risk for developing a complement-associated disorder” (e.g., an AP-associated disorder or a CP-associated disorder) are not all the subjects within a species of interest.

A subject “suspected of having a complement-associated disorder” (e.g., an alternative complement pathway-associated disorder) is one having one or more (e.g., two, three, four, five, six, seven, eight, nine, or 10 or more) symptoms of the disease. Symptoms of these disorders will vary depending on the particular disorder, but are known to those of skill in the art of medicine. For example, symptoms of DDD include, e.g.: one or both of hematuria and proteinuria; acute nephritic syndrome; drusen development and/or visual impairment; acquired partial lipodystrophy and complications thereof; and the presence of serum C3 nephritic factor (C3NeF), an autoantibody directed against C3bBb, the C3 convertase of the alternative complement pathway. (See, e.g., Appel et al. (2005), supra). Symptoms of aHUS include, e.g., severe hypertension, proteinuria, uremia, lethargy/fatigue, irritability, thrombocytopenia, microangiopathic hemolytic anemia, and renal function impairment (e.g., acute renal failure). Symptoms of TTP include, e.g., microthrombi, thrombocytopenia, fever, low ADAMTS 13 metalloproteinase expression or activity, fluctuating central nervous system abnormalities, renal failure, microangiopathic hemolytic anemia, bruising, purpura, nausea and vomiting (e.g., resulting from ischemia in the GI tract or from central nervous system involvement), chest pain due to cardiac ischemia, seizures, and muscle and joint pain. Symptoms of RA can include, e.g., stiffness, swelling, fatigue, anemia, weight loss, fever, and often, crippling pain. Some common symptoms of rheumatoid arthritis include joint stiffness upon awakening that lasts an hour or longer; swelling in a specific finger or wrist joints; swelling in the soft tissue around the joints; and swelling on both sides of the joint. Swelling can occur with or without pain, and can worsen progressively or remain the same for years before progressing. Symptoms of HELLP are known in the art of medicine and include, e.g., malaise, epigastric pain, nausea, vomiting, headache, right upper quadrant pain, hypertension, proteinuria, blurred vision, gastrointestinal bleeding, hypoglycemia, paresthesia, elevated liver enzymes/liver damage, anemia (hemolytic anemia), and low platelet count, any of which in combination with pregnancy or recent pregnancy. (See, e.g., Tomsen (1995) Am J Obstet Gynecol 172:1876-1890; Sibai (1986) Am J Obstet Gynecol 162:311-316; and Padden (1999), supra.) Symptoms of PNH include, e.g., hemolytic anemia (a decreased number of red blood cells), hemoglobinuria (the presence of hemoglobin in the urine particularly evident after sleeping), and hemoglobinemia (the presence of hemoglobin in the bloodstream). PNH-afflicted subjects are known to have paroxysms, which are defined here as incidences of dark-colored urine, dysphagia, fatigue, erectile dysfunction, thrombosis, and recurrent abdominal pain.

Symptoms of CAPS are well known in the art of medicine and include, e.g., histopathological evidence of multiple small vessel occlusions; the presence of antiphospholipid antibodies (usually at high titer), vascular thromboses, severe multi-organ dysfunction, malignant hypertension, acute respiratory distress syndrome, disseminated intravascular coagulation, microangiopathic hemolytic anemia, schistocytes, and thrombocytopenia. CAPS can be distinguished from APS in that patients with CAPS generally present with severe multiple organ dysfunction or failure, which is characterized by rapid, diffuse small vessel ischemia and thromboses predominantly affecting the parenchymal organs. In contrast, APS is associated with single venous or arterial medium-to-large blood vessel occlusions. Symptoms of MG include, e.g., fatigability and a range of muscle weakness-related conditions including: ptosis (of one or both eyes), diplopia, unstable gait, depressed or distorted facial expressions, and difficulty chewing, talking, or swallowing. In some instances, a subject can present with partial or complete paralysis of the respiratory muscles. Symptoms of CAD include, e.g., pain, fever, pallor, anemia, reduced blood flow to the extremities (e.g., with gangrene), and renal disease or acute renal failure. In some embodiments, the symptoms can occur following exposure to cold temperatures.

From the above it will be clear that subjects “suspected of having a complement-associated disorder” are not all the subjects within a species of interest.

In some embodiments, the methods can include identifying the subject as one having, suspected of having, or at risk for developing, a complement-associated disorder in a subject. Suitable methods for identifying the subject are known in the art. For example, suitable methods (e.g., sequencing techniques or use of microarrays) for determining whether a human subject has a DDD-associated mutation in a CFH, CFHR5, or C3 gene are described in, e.g., Licht et al. (2006) Kidney Int 70:42-50; Zipfel et al. (2006), supra; Ault et al. (1997) J Biol Chem 272:25168-75; Abrera-Abeleda et al. (2006) J Med Genet. 43:582-589; Poznansky et al. (1989) J Immunol 143:1254-1258; Jansen et al. (1998) Kidney Int 53:331-349; and Hegasy et al. (2002) Am J Pathol 161:2027-2034. Methods for detecting the presence of characteristic DDD-associated electron-dense deposits are also well known in the art. For example, a medical practitioner can obtain a tissue biopsy from the kidney of a patient and subject the tissue to electron microscopy. The medical practitioner may also examine the tissue by immunofluorescence to detect the presence of C3 using an anti-C3 antibody and/or light microscopy to determine if there is membranoproliferative glomerulonephritis. See, e.g., Walker et al. (2007) Mod Pathol 20:605-616 and Habib et al. (1975) Kidney Int 7:204-215. In some embodiments, the identification of a subject as one having DDD can include assaying a blood sample for the presence of C3NeF. Methods for detecting the presence of C3NeF in blood are described in, e.g., Schwertz et al. (2001) Pediatr Allergy Immunol 12:166-172.

In some embodiments, the medical practitioner can determine whether there is increased complement activation in a subject's serum. Indicia of increased complement activation include, e.g., a reduction in CH50, a decrease in C3, and an increase in C3dg/C3d. See, e.g., Appel et al. (2005), supra. In some embodiments, a medical practitioner can examine a subject's eye for evidence of the development of drusen and/or other visual pathologies such as AMD. For example, a medical practitioner can use tests of retinal function such as, but not limited to, dark adaptation, electroretinography, and electrooculography (see, e.g., Colville et al. (2003) Am J Kidney Dis 42:E2-5).

Methods for identifying a subject as one having, suspected of having, or at risk for developing, TTP are also known in the art. For example, Miyata et al. describe a variety of assays for measuring ADAMTS13 activity in a biological sample obtained from a subject (Curr Opin Hematol (2007) 14(3):277-283). Suitable ADAMTS13 activity assays, as well as phenotypically normal ranges of ADAMTS 13 activity in a human subject, are described in, e.g., Tsai (2003) J Am Soc Nephrol 14:1072-1081; Furlan et al. (1998) New Engl J Med 339:1578-1584; Matsumoto et al. (2004) Blood 103:1305-1310; and Mori et al. (2002) Transfusion 42:572-580. Methods for detecting the presence of inhibitors of ADAMTS13 (e.g., autoantibodies that bind to ADAMTS13) in a biological sample obtained from a subject are known in the art. For example, a serum sample from a patient can be mixed with a serum sample from a subject without TTP to detect the presence of anti-ADAMTS13 antibodies. In another example, immunoglobulin protein can be isolated from patient serum and used in in vitro ADAMTS13 activity assays to determine if an anti-ADAMTS13 antibody is present. See, e.g., Dong et al. (2008) Am J Hematol 83(10):815-817. In some embodiments, risk of developing TTP can be determined by assessing whether a patient carries one or more mutations in the ADAMTS13 gene. Suitable methods (e.g., nucleic acid arrays or DNA sequencing) for detecting a mutation in the ADAMTS13 gene are known in the art and described in, e.g., Levy et al., supra; Kokame et al., supra; Licht et al., supra; and Noris et al., supra.

In addition, methods for identifying a subject as one having, suspected of having, or at risk for developing aHUS are known in the art. For example, laboratory tests can be performed to determine whether a human subject has thrombocytopenia, microangiopathic hemolytic anemia, or acute renal insufficiency. Thrombocytopenia can be diagnosed by a medical professional as one or more of: (i) a platelet count that is less than 150,000/mm³ (e.g., less than 60,000/mm³); (ii) a reduction in platelet survival time, reflecting enhanced platelet disruption in the circulation; and (iii) giant platelets observed in a peripheral smear, which is consistent with secondary activation of thrombocytopoiesis. Microangiopathic hemolytic anemia can be diagnosed by a medical professional as one or more of: (i) hemoglobin concentrations that are less than 10 mg/dL (e.g., less than 6.5 mg/dL); (ii) increased serum lactate dehydrogenase (LDH) concentrations (>460 U/L); (iii) hyperbilirubinemia, reticulocytosis, circulating free hemoglobin, and low or undetectable haptoglobin concentrations; and (iv) the detection of fragmented red blood cells (schistocytes) with the typical aspect of burr or helmet cells in the peripheral smear together with a negative Coombs test. (See, e.g., Kaplan et al. (1992) “Hemolytic Uremic Syndrome and Thrombotic Thrombocytopenic Purpura,” Informa Health Care (ISBN 0824786637) and Zipfel (2005) “Complement and Kidney Disease,” Springer (ISBN 3764371668).)

A subject can also be identified as having aHUS by evaluating blood concentrations of C3 and C4 as a measure of complement activation or dysregulation. In addition, as is clear from the foregoing disclosure, a subject can be identified as having genetic aHUS by identifying the subject as harboring one or more mutations in a gene associated with aHUS such as CFI, CFB, CFH, or MCP (supra). Suitable methods for detecting a mutation in a gene include, e.g., DNA sequencing and nucleic acid array techniques. (See, e.g., Breslin et al. (2006) Clin Am Soc Nephrol 1:88-99 and Goicoechea de Jorge et al. (2007) Proc Natl Acad Sci USA 104:240-245.)

Methods for diagnosing a subject as one having, suspected of having, or at risk for developing, RA are also known in the art of medicine. For example, a medical practitioner can examine the small joints of the hands, wrists, feet, and knees to identify inflammation in a symmetrical distribution. The practitioner may also perform a number of tests to exclude other types of joint inflammation including arthritis due to infection or gout. In addition, rheumatoid arthritis is associated with abnormal antibodies in the blood circulation of afflicted patients. For example, an antibody referred to as “rheumatoid factor” is found in approximately 80% of patients. In another example, anti-citrulline antibody is present in many patients with rheumatoid arthritis and thus it is useful in the diagnosis of rheumatoid arthritis when evaluating patients with unexplained joint inflammation. See, e.g., van Venrooij et al. (2008) Ann NY Acad Sci 1143:268-285 and Habib et al. (2007) Immunol Invest 37(8):849-857. Another antibody called “the antinuclear antibody” (ANA) is also frequently found in patients with rheumatoid arthritis. See, e.g., Benucci et al. (2008) Clin Rheumatol 27(1):91-95; Julkunen et al. (2005) Scan J Rheumatol 34(2):122-124; and Miyawaki et al. (2005) J Rheumatol 32(8):1488-1494.

A medical practitioner can also examine red blood cell sedimentation rate to help in diagnosing RA in a subject. The sedimentation rate can be used as a crude measure of the inflammation of the joints and is usually faster during disease flares and slower during remissions. Another blood test that can be used to measure the degree of inflammation present in the body is the C-reactive protein.

Furthermore, joint x-rays can also be used to diagnose a subject as having rheumatoid arthritis. As RA progresses, the x-rays can show bony erosions typical of rheumatoid arthritis in the joints. Joint x-rays can also be helpful in monitoring the progression of disease and joint damage over time. Bone scanning, a radioactive test procedure, can demonstrate the inflamed joints.

Methods for identifying a subject as one having, suspected of having, or at risk for developing, HELLP are known in the art of medicine. Hallmark symptoms of HELLP syndrome include hemolysis, elevated liver enzymes, and low platelet count. Thus, a variety of tests can be performed on blood from a subject to determine the level of hemolysis, the concentration of any of a variety of liver enzymes, and the platelet level in the blood. For example, the presence of schistocytes and/or elevated free hemoglobin, bilirubin, or serum LDH levels is an indication of intravascular hemolysis. Routine laboratory testing can be used to determine the platelet count as well as the blood level of liver enzymes such as, but not limited to, aspartate aminotransferase (AST) and alanine transaminase (ALT). Suitable methods for identifying a subject as having HELLP syndrome are also described in, e.g., Sibai et al. (1993), supra; Martin et al. (1990), supra; Padden (1999), supra; and Gleicher and Buttino (1998) “Principles & Practice of Medical Therapy in Pregnancy,” 3^{rd} Edition, Appleton & Lange (ISBN 083857677X).

Methods for identifying a subject as having, suspected of having, or at risk for developing PNH are known in the art of medicine. The laboratory evaluation of hemolysis normally includes hematologic, serologic, and urine tests. Hematologic tests include an examination of the blood smear for morphologic abnormalities of red blood cells (RBC), and the measurement of the reticulocyte count in whole blood (to determine bone marrow compensation for RBC loss). Serologic tests include lactate dehydrogenase (LDH; widely performed), and free hemoglobin (not widely performed) as a direct measure of hemolysis. LDH levels, in the absence of tissue damage in other organs, can be useful in the diagnosis and monitoring of patients with hemolysis. Other serologic tests include bilirubin or haptoglobin, as measures of breakdown products or scavenging reserve, respectively. Urine tests include bilirubin, hemosiderin, and free hemoglobin, and are generally used to measure gross severity of hemolysis and for differentiation of intravascular vs. extravascular etiologies of hemolysis rather than routine monitoring of hemolysis. Further, RBC numbers, RBC hemoglobin, and hematocrit are generally performed to determine the extent of any accompanying anemia.

Suitable methods for identifying the subject as having MG can be qualitative or quantitative. For example, a medical practitioner can examine the status of a subject's motor functions using a physical examination. Other qualitative tests include, e.g., an ice-pack test, wherein an ice pack is applied to a subject's eye (in a case of ocular MG) to determine if one or more symptoms (e.g., ptosis) are improved by cold (see, e.g., Sethi et al. (1987) Neurology 37(8):1383-1385). Other tests include, e.g., the “sleep test,” which is based on the tendency for MG symptoms to improve following rest. In some embodiments, quantitative or semi-quantitative tests can be employed by a medical practitioner to determine if a subject has, is suspected of having, or is at risk for developing, MG. For example, a medical practitioner can perform a test to detect the presence or amount of MG-associated autoantibodies in a serum sample obtained from a subject. MG-associated autoantibodies include, e.g., antibodies that bind to, and modulate the activity of, acetylcholine receptor (AChR), muscle-specific receptor tyrosine kinase (MuSK), and/or striational protein. (See, e.g., Conti-Fine et al. (2006), supra.) Suitable assays useful for detecting the presence or amount of an MG-associated antibody in a biological sample are known in the art and described in, e.g., Hoch et al. (2001) Nat Med 7:365-368; Vincent et al. (2004) Semin Neurol 24:125-133; McConville et al. (2004) Ann Neurol 55:580-584; Boneva et al. (2006) J Neuroimmunol 177:119-131; and Romi et al. (2005) Arch Neurol 62:442-446.

Additional methods for diagnosing MG include, e.g., electrodiagnostic tests (e.g., single-fiber electromyography) and the Tensilon (or edrophonium) test, which involves injecting a subject with the acetylcholinesterase inhibitor edrophonium and monitoring the subject for an improvement in one or more symptoms. See, e.g., Pascuzzi (2003) Semin Neurol 23(1):83-88; Katirji et al. (2002) Neurol Clin 20:557-586; and “Guidelines in Electrodiagnostic Medicine. American Association of Electrodiagnostic Medicine,” Muscle Nerve 15:229-253.

A subject can be identified as having CAD using an assay to detect the presence or amount (titer) of agglutinating autoantibodies that bind to the I antigen on red blood cells. The antibodies can be monoclonal (e.g., monoclonal IgM or IgA) or polyclonal. Suitable methods for detecting these antibodies are described in, e.g., Christenson and Dacie (1957) Br J Haematol 3:153-164 and Christenson et al. (1957) Br J Haematol 3:262-275. A subject can also be diagnosed as having CAD using one or more of a complete blood cell count (CBC), urinalysis, biochemical studies, and a Coombs test to test for hemolysis in blood. For example, biochemical studies can be used to detect elevated lactase dehydrogenase levels, elevated unconjugated bilirubin levels, low haptoglobin levels, and/or the presence of free plasma hemoglobin, all of which can be indicative of acute hemolysis. Other tests that can be used to detect CAD include detecting complement levels in the serum. For example, due to consumption during the acute phase of hemolysis, measured plasma complement levels (e.g., C2, C3, and C4) are decreased in CAD. Typical (or infectious) HUS, unlike aHUS, is often identifiable by a prodrome of diarrhea, often bloody in nature, which results from infection with a shiga-toxin producing microorganism. A subject can be identified as having typical HUS when shiga toxins and/or serum antibodies against shiga toxin or LPS are detected in the stool of an individual. Suitable methods for testing for anti-shiga toxin antibodies or LPS are known in the art. For example, methods for detecting antibodies that bind to shiga toxins Stx1 and Stx2 or LPS in humans are described in, e.g., Ludwig et al. (2001) J Clin Microbiol 39(6):2272-2279.

In some embodiments, a C5-binding polypeptide described herein can be administered to a subject as a monotherapy. Alternatively, as described above, the antibody can be administered to a subject as a combination therapy with another treatment, e.g., another treatment for DDD, TTP, wet or dry AMD, aHUS, PNH, RA, HELLP, MG, CAD, CAPS, tHUS, asthma, COPD, or any other complement-associated disorder known in the art or described herein. For example, the combination therapy can include administering to the subject (e.g., a human patient) one or more additional agents (e.g., anti-coagulants, anti-hypertensives, or corticosteroids) that provide a therapeutic benefit to the subject who has, or is at risk of developing, DDD. In some embodiments, the combination therapy can include administering to the subject (e.g., a human patient) a C5-binding polypeptide and an immunosuppressive agent such as Remicade® for use in treating RA. In some embodiments, the C5-binding polypeptide and the one or more additional active agents are administered at the same time. In other embodiments, a C5-binding polypeptide is administered first in time and the one or more additional active agents are administered second in time. In some embodiments, the one or more additional active agents are administered first in time and the C5-binding polypeptide is administered second in time.

A C5-binding polypeptide described herein can replace or augment a previously or currently administered therapy. For example, upon treating with a C5-binding polypeptide, administration of the one or more additional active agents can cease or diminish, e.g., be administered at lower levels. In some embodiments, administration of the previous therapy can be maintained. In some embodiments, a previous therapy will be maintained until the level of the C5-binding polypeptide reaches a level sufficient to provide a therapeutic effect. The two therapies can be administered in combination.

Monitoring a subject (e.g., a human patient) for an improvement in a complement-associated disorder, as defined herein, means evaluating the subject for a change in a disease parameter, e.g., an improvement in one or more symptoms of the disease (e.g., an improvement in one or more symptoms of a pulmonary disorder). Such symptoms include any of the symptoms of complement-associated disorders known in the art and/or described herein. In some embodiments, the evaluation is performed at least 1 hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration. The subject can be evaluated in one or more of the following periods: prior to beginning of treatment; during the treatment; or after one or more elements of the treatment have been administered. Evaluating can include evaluating the need for further treatment, e.g., evaluating whether a dosage, frequency of administration, or duration of treatment should be altered. It can also include evaluating the need to add or drop a selected therapeutic modality, e.g., adding or dropping any of the treatments for any of the complement-associated disorders described herein.

Ex Vivo Approaches.

An ex vivo strategy for treating or preventing a complement-associated disorder (e.g., an AP-associated disorder or a CP-associated disorder) can involve transfecting or transducing one or more cells obtained from a subject with a polynucleotide encoding a C5-binding polypeptide described herein.

The transfected or transduced cells are then returned to the subject. The cells can be any of a wide range of types including, without limitation, hemopoietic cells (e.g., bone marrow cells, macrophages, monocytes, dendritic cells, T cells, or B cells), fibroblasts, epithelial cells, endothelial cells, keratinocytes, or muscle cells. Such cells can act as a source (e.g., sustained or periodic source) of the C5-binding polypeptide for as long as they survive in the subject. In some embodiments, the vectors and/or cells can be configured for inducible or repressible expression of the C5-binding polypeptide (see, e.g., Schockett et al. (1996) Proc Natl Acad Sci USA 93: 5173-5176 and U.S. Pat. No. 7,056,897).

Preferably, the cells are obtained from the subject (autologous), but can potentially be obtained from a subject of the same species other than the subject (allogeneic).

Suitable methods for obtaining cells from a subject and transducing or transfecting the cells are known in the art of molecular biology. For example, the transduction step can be accomplished by any standard means used for ex vivo gene therapy, including calcium phosphate, lipofection, electroporation, viral infection (see above), and biolistic gene transfer. (See, e.g., Sambrook et al. (supra) and Ausubel et al. (1992) “Current Protocols in Molecular Biology,” Greene Publishing Associates.) Alternatively, liposomes or polymeric microparticles can be used. Cells that have been successfully transduced can be selected, for example, for expression of the coding sequence or of a drug resistance gene.

### Therapeutic Kits

The disclosure also features therapeutic and diagnostic kits containing, among other things, one or more of the C5-binding polypeptides described herein. The therapeutic kits can contain, e.g., a suitable means for delivery of one or more C5-binding polypeptides to a subject. In some embodiments, the means is suitable for subcutaneous delivery of the antibody or antigen-binding fragment thereof to the subject. The means can be, e.g., a syringe or an osmotic pump.

In some embodiments, the means is suitable for intrapulmonary delivery of a C5-binding polypeptide to a subject, e.g., for use in treatment or prevention of a complement-associated pulmonary disorder such as, but not limited to, COPD or asthma. Accordingly, the means can be, e.g., an oral or nasal inhaler (see above).

The inhaler can be, e.g., a metered dose inhaler (MDI), dry powder inhaler (DPI), or a nebulizer. Such a kit can also, optionally, include instructions for administering (e.g., self-administration of) the C5-binding polypeptide to a subject.

The therapeutic kits can include, e.g., one or more additional active agents for treating or preventing a complement-associated disorder and/or ameliorating a symptom thereof. For example, therapeutic kits designed for use in treating or preventing a complement-associated pulmonary disorder can include one or more additional active agents including, but not limited to, another antibody therapeutic (e.g., an anti-IgE antibody, an anti-IL-4 antibody, or an anti-IL-5 antibody), a small molecule anti-IgE inhibitor (e.g., montelukast sodium), a sympathomimetic (e.g., albuterol), an antibiotic (e.g., tobramycin), a deoxyribonuclease (e.g., pulmozyme), an anticholinergic drug (e.g., ipratropium bromide), a corticosteroid (e.g., dexamethasone), a β-adrenoreceptor agonist, a leukotriene inhibitor (e.g., zileuton), a 5-lipoxygenase inhibitor, a phosphodiesterase (PDE) inhibitor, a CD23 antagonist, an IL-13 antagonist, a cytokine release inhibitor, a histamine H1 receptor antagonist, an anti-histamine, an anti-inflammatory agent (e.g., cromolyn sodium or any other anti-inflammatory agent known in the art or described herein), or a histamine release inhibitor.

In some embodiments, the means can be suitable for administration of a C5-binding polypeptide described herein to the eye of a subject in need thereof, e.g., a subject afflicted with AMD. The means can be, e.g., a syringe, a trans-scleral patch, or even a contact lens containing the polypeptide. The means can, in some embodiments, be an eye dropper, wherein the C5-binding polypeptide is formulated for such administration. The means can also be, e.g., a contact lens case in embodiments in which, e.g., the C5-binding polypeptide is formulated as part of a contact lens hydrating, cleaning, or soaking solution. Such therapeutic kits can also include, e.g., one or more additional therapeutic agents for use in treating complement-associated disorder of the eye. The therapeutic agents can be, e.g., bevacizumab or the Fab fragment of bevacizumab, ranibizumab, both sold by Roche Pharmaceuticals, Inc., pegaptanib sodium (Mucogen®; Pfizer, Inc.), and verteporfin (Visudyne®; Novartis). Such a kit can also, optionally, include instructions for administering the C5-binding polypeptide to a subject.

In some embodiments, the means can be suitable for intraarticular administration of a C5-binding polypeptide described herein to a subject in need thereof, e.g., a subject afflicted with RA. The means can be, e.g., a syringe or a double-barreled syringe. See, e.g., U.S. Pat. Nos. 6,065,645 and 6,698,622. A double-barreled syringe is useful for administering to a joint two different compositions with only one injection. Two separate syringes may be incorporated for use in administering the therapeutic while drawing off knee fluid for analysis (tapping) in a push-pull fashion. Additional therapeutic agents that can be administered with the C5-binding polypeptide in conjunction with the double-barreled syringe, or which can otherwise be generally included in the therapeutic kits described herein, include, e.g., NSAIDs, corticosteroids, methotrexate, hydroxychloroquine, anti-TNF agents such as etanercept and infliximab, a B cell depleting agent such as rituximab, an interleukin-1 antagonist, or a T cell costimulatory blocking agent such as abatacept. Such a kit can also, optionally, include instructions for administering a C5-binding polypeptide to a subject.

The following examples are intended to illustrate, not limit, the invention.

### Example 1

### The R38Q Substitution does not Significantly Affect Binding to C5

The kinetics of binding between complement component C5 and either pexelizumab (discussed supra) or a variant of pexelizumab were studied using the Biacore™ 3000 system (Biacore, GE Healthcare). The pexelizumab variant comprises the following amino acid sequence:

(SEQ ID NO: 2)
DIQMTQSPSSLSASVGDRVTITCGASENIYGALNWYQQKPGKAPKLLI
YGATNLADGVPSRFSGSGSGTDFTLTISSLQPEDFATYYCQNVLNTPL
TFGQGTKVEIKRTGGGGSGGGGSGGGGSQVQLVQSGAEVKKPGASVKV
SCKASGYIFSNYWIQWVRQAPGQGLEWMGEILPGSGSTEYTENFKDRV
TMTRDTSTSTVYMELSSLRSEDTAVYYCARYFFGSSPNWYFDVWGQGT
LVTVSS.

The amino acid sequence of the variant differs from the amino acid sequence of pexelizumab by two amino acids. First, the variant single chain antibody does not contain an amino-terminal alanine that is present in pexelizumab. The variant antibody also contains a substitution of the arginine (R) at position 38 of pexelizumab for glutamine (Q) (in bold above). Hereinafter, the variant single chain antibody is referred to as “R38Q scFv.”

Human C5 protein was obtained from Advanced Research Technologies (catalogue no. A120; Montreal, Quebec, Canada). R38Q scFv was prepared in a 1.9 mg/mL solution containing 0.01% Tween-80. The binding kinetics between R38Q scFv and C5 were measured by directly immobilizing the antibody to a CMS sensor chip (Biacore, GE Healthcare). All measurements were performed at a 25° C. sensor surface temperature.

Various concentrations of C5 were passed over the chip surface containing bound R38Q scFv. Concentrations of 0.1875 nM to 24 nM C5 were evaluated with a dissociation time of 1,500 seconds. The binding kinetics between R38Q scFv and C5 were determined using a 1:1 Langmuir model (the kinetics data are set forth in Table 1). After fitting the data to the Langmuir model, the K_{D} of the interaction between R38Q scFv and C5 was determined to be 108 μM. Under similar conditions, the K_{D} of the interaction between pexelizumab and C5 was determined to be 390 μM. These data indicated that the R38Q substitution does not significantly affect the ability of the R38Q scFv antibody to bind to C5.

TABLE 1

| Experiment | kₐ (M⁻¹s⁻¹) | k_{d} (s⁻¹) | K_{D} (M) | Residues | χ² |
|---|---|---|---|---|---|
| R38Q scFv | 5.59e5 | 6.06e−5 | 1.08e−10 | ±2.0 | 0.227 |
| and C5 | | | | | |
| R38Q scFv | 334 | 0.0107 | 3.2e−5 | ±3.5 | 0.621 |
| self | | | | | |
| association | | | | | |

The self-association of the R38Q scFv was also evaluated using Biacore. Briefly, R38Q scFv was directly immobilized on the CM5 sensor chip and various concentrations (0.6-75 μM) of R38Q scFv were passed over the chip surface. Although the data obtained in the self-association data did not fit the Langmuir model per se (χ² of 0.621 and residues of ±3.5), the K_{D} was determined to be 32 μM, which was comparable to previous studies with pexelizumab under similar conditions.

### Example 2

### The R38Q Substitution does not Significantly Affect Inhibition of Hemolysis

Pexelizumab is a potent inhibitor of hemolysis in vitro. To determine if the R38Q substitution affects the ability of the R38Q scFv antibody to inhibit hemolysis, the antibody was evaluated in an in vitro red blood cell hemolysis assay.

The red blood cell hemolysis assay is generally described in detail in, e.g., Rinder et al. (1995) J Clin Invest 96:1564-1572. Briefly, normal human serum was added to multiple wells of a 96 well assay plate such that the concentration of the serum in each well was approximately 10%. Different concentrations (20, 10, 5, 2.5, 1, and 0.5 μg/mL) of pexelizumab or the R38Q substituted antibody were added to serum-containing wells. Some of the serum-containing wells did not contain antibody and served as negative controls. Chicken erythrocytes (Lampire Biological Laboratories, Piperville, Pa.) were washed and resuspended in buffer at a final concentration of 5×10⁷ cells/mL. The erythrocytes were sensitized to lysis by incubating the cells with an anti-chicken red blood cell polyclonal antibody composition. The sensitized erythrocytes were added to the wells of the 96 well plate and the plate was incubated at 37° C. for 30 minutes. Hemoglobin release was measured by apparent absorbance at 415 nm using a microplate reader.

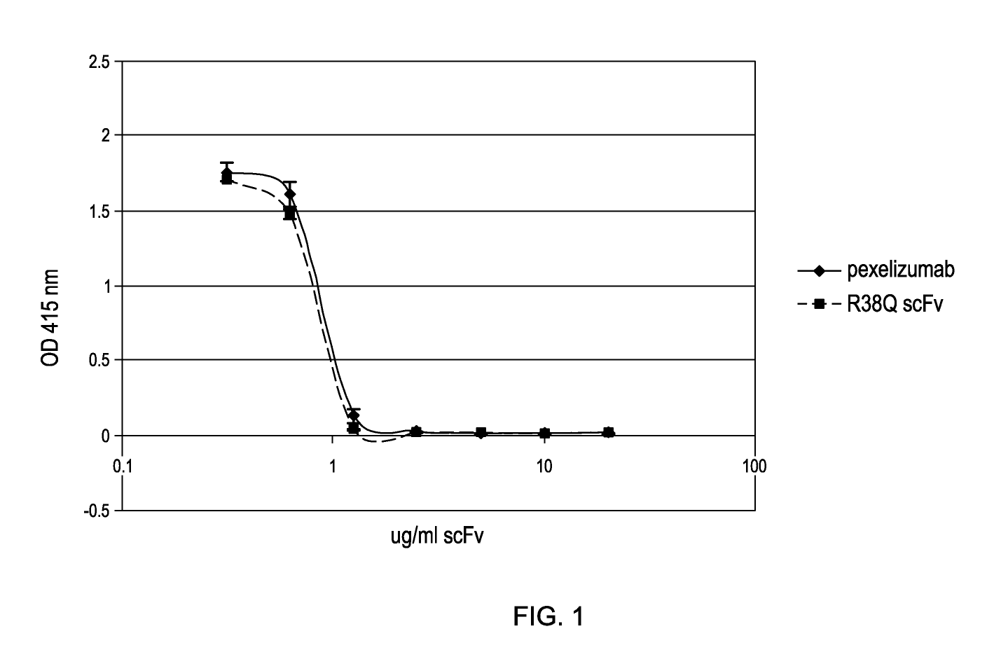

As shown in FIG. 1, both pexelizumab and the R38Q substituted antibody inhibited erythrocyte hemolysis, each having an IC₅₀ of approximately 2 μg/mL. These results indicate that the R38Q substitution does not affect the ability of the R38Q scFv to inhibit hemolysis in vitro.

### Example 3

### R38Q scFv Exhibits Enhanced Solubility as Compared to Pexelizumab

The solubility of R38Q scFv was evaluated. Different amounts of R38Q scFv were added to a phosphate-buffered solution (10 mM sodium phosphate, 150 mM NaCl, pH 7). R38Q scFv solutions of up to 50 mg/mL could be prepared in the buffer. In contrast, the solubility limit of pexelizumab in the same buffer was approximately 2 mg/mL. These results indicated that the R38Q substitution increased the solubility of the variant antibody in aqueous solution.

### Example 4

### R38Q scFv Reversibly Oligomerizes in Solution at High Concentration

Protein oligomerization is a significant risk factor for high concentration solutions of proteins (e.g., antibodies) and oligomerization can affect the activity of a biologically active protein. See, e.g., Treuheit et al. (2002) Pharm Res 19(4):511-516 and Shire et al. (2004) J Pharm Sci 93:1390-1402. To characterize the extent of oligomerization (if at all) of R38Q scFv in solution at high concentrations, several solutions (1.9 mg/mL, 10 mg/mL, and 50 mg/mL) of the antibody were prepared in phosphate buffer (10 mM sodium phosphate, 150 mM sodium chloride, pH 7). The oligomerization state of R38Q scFv in solution was analyzed by subjecting 20 μg of protein from each solution to size exclusion chromatography (SEC) high-performance liquid chromatography (HPLC). The results of the experiments are summarized in Table 2. (The dimeric form of R38Q scFv is the dominant form of the antibody in solution.) These results indicated that R38Q scFv forms oligomeric species in solution and that the percentage of oligomeric species in solution increases with concentration.

TABLE 2

| | % Oligomeric Form* | | | | | | | |
|---|---|---|---|---|---|---|---|---|
| Sample | Monomer | Dimer | Trimer | Tetramer | Pentamer | Hexamer | Heptamer | Octomer |
| 1.9 mg/mL | 4.82 | 77.28 | 13.09 | 2.94 | 0.89 | ND | ND | ND |
| 10 mg/mL | 2.59 | 61.46 | 19.89 | 8.54 | 3.99 | 3.53 | ND | ND |
| **50 mg/mL | 1.32 | 30.02 | 15.27 | 9.94 | 7.38 | 5.92 | 4.81 | 3.84 |

*The percentage of each form of R38Q scFv is calculated as the percent area.
**The 50 mg/mL solution also contained approximately 21.5% higher order oligomers.
ND means “not detected.”

To determine if the concentration-dependent oligomerization of R38Q scFv in solution is reversible, the following experiment was performed. First, a 50 mg/mL solution of R38Q scFv was prepared in the following buffer: 10 mM sodium phosphate pH 7, 150 mM sodium chloride, and 0.01% Tween 20. The 50 mg/mL solution was then diluted to 2 mg/mL and incubated for various times (108, 1100, 5762 minutes) at 4-5° C. before subjecting 20 μg of the 2 mg/mL sample to SEC HPLC. The results of the experiment are summarized in Table 3.

TABLE 3

| Time at | % Oligomeric Form* | | | | | | | | |
|---|---|---|---|---|---|---|---|---|---|
| 2 mg/ml | | | | | | | | | Higher |
| (min) | Monomer | Dimer | Trimer | Tetramer | Pentamer | Hexamer | Heptamer | Octomer | Order |
| 0 | 1.15 | 30.57 | 15.44 | 9.81 | 7.54 | 5.83 | 4.70 | 4.04 | 20.93 |
| 108 | 3.95 | 35.34 | 19.93 | 13.99 | 10.35 | 7.65 | 8.72 | ND | ND |
| 1100 | 5.59 | 51.29 | 25.68 | 11.45 | 4.30 | 1.61 | ND | ND | ND |
| 5762 | 4.65 | 73.53 | 16.68 | 3.84 | 1.22 | ND | ND | ND | ND |

*The percentage of each form of R38Q scFv is calculated as the percent area.
ND means “not detected.”

Upon dilution and over time, the higher order oligomeric forms of R38Q scFv detected in the 50 mg/mL solution dissociate into lower order species. For example, after 5,762 minutes, no hexameric, heptameric, octomeric, or higher order species were detected in the diluted 2 mg/mL solution. In fact, the percentage of the dominant, dimeric form 5762 minutes after diluting to a 2 mg/mL solution (73.53%) was approximately the same amount that was present in the undiluted 1.9 mg/mL solution analyzed above (77.28%; see Table 2). These results indicate that the concentration-dependent oligomerization of R38Q scFv in solution is reversible. The results also indicate that the multimeric and higher order oligomeric forms of R38Q scFv present in a high concentration solution, when diluted prior to administration or when diluted upon administration to a subject, are likely to dissociate into the predominant dimeric form.

### Example 5

### High Concentration R38Q scFv Formulation does not Significantly Affect Antibody Activity

As noted above, oligomerization of biologically active proteins can, in some cases, affect the biological activity of the protein. To determine whether reversible oligomerization of R38Q scFv affects its biological activity, several solutions (1.9 mg/mL, 10 mg/mL, and 50 mg/mL) of the antibody were prepared in phosphate buffer (10 mM sodium phosphate, 150 mM sodium chloride, pH 7) as described above and evaluated in an in vitro hemolysis assay (see above).

Normal human serum was added to multiple wells of a 96 well assay plate. R38Q scFv protein from the 50 mg/mL solution was added to a set of the serum-containing wells in an amount such that the final concentration of the antibody in the well was 10, 5, 2.5, 1.25, 0.75, 0.375, or 0.188 μg/mL, respectively. R38Q scFv antibody protein from the 10 mg/mL and 1.9 mg/mL solutions was also added to parallel sets of serum-containing wells in amounts sufficient to achieve the same final concentrations of antibody in the wells. Some of the serum-containing wells did not contain antibody and served as negative controls.

Sensitized erythrocytes were then added to the wells of the 96 well plate and the plate was incubated at 37° C. for 30 minutes. Hemoglobin release was measured by apparent absorbance at 415 nm using a microplate reader.

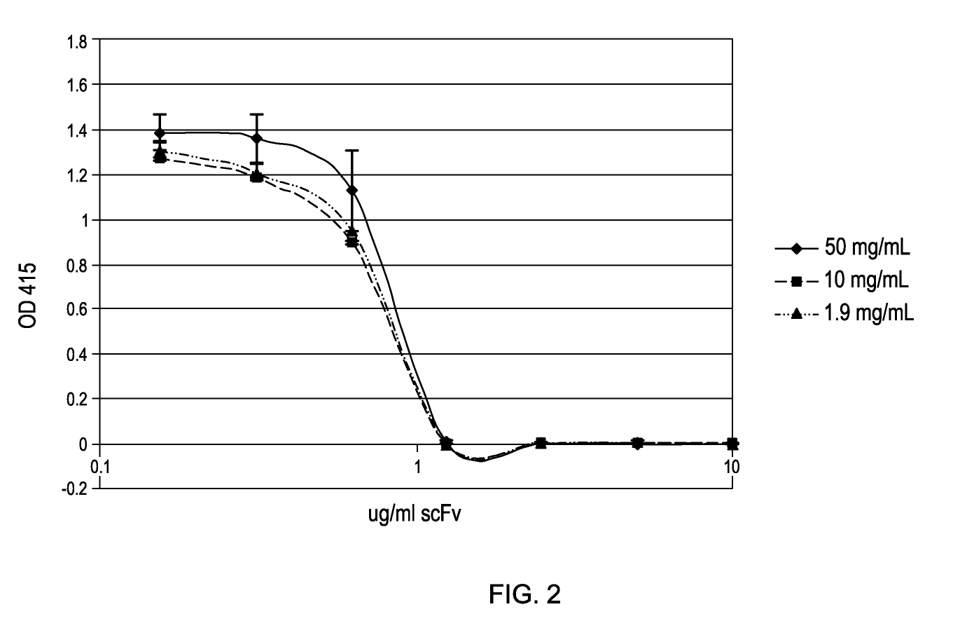

As shown in FIG. 2, the reversible concentration-dependent oligomerization of R38Q scFv protein did not significantly affect the ability of the antibody to inhibit in vitro hemolysis of the chicken red blood cells. These results indicate that the R38Q scFv protein present in multimeric and higher order oligomeric forms in high concentration solutions retains biological activity. The results also indicate that when diluted prior to administration or when diluted upon administration to a subject, the R38Q scFv protein present in high concentration solutions is competent to therapeutically inhibit hemolysis in the subject.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

1. A polypeptide comprising: (i) amino acids 1-107 depicted in SEQ ID NO:2 and (ii) amino acids 125-246 depicted in SEQ ID NO:2, with the proviso that the polypeptide is not a whole antibody.

2. A polypeptide comprising an amino acid sequence that is at least 80% identical to an amino acid sequence comprising: (i) amino acids 1-107 depicted in SEQ ID NO:2 and (ii) amino acids 125-246 depicted in SEQ ID NO:2, wherein the polypeptide binds to human complement component C5 and the amino acid sequence of the polypeptide comprises the glutamine residue at position 38 of SEQ ID NO:2, with the proviso that the polypeptide is not a whole antibody.

3. (canceled)

4. A polypeptide that comprises at least 50 contiguous amino acids of SEQ ID NO:2, wherein the polypeptide binds to human complement component C5 and the at least 50 amino acids comprise the glutamine residue at position 38 of SEQ ID NO:2, with the proviso that the polypeptide is not a whole antibody.

5. The polypeptide of claim 1, wherein the polypeptide comprises the amino acid sequence depicted in SEQ ID NO:2.

6. (canceled)

7. A fusion polypeptide comprising:
(a) the polypeptide of claim 1; and
(b) an amino acid sequence that is heterologous to amino acids 1-107 and 125-246 of SEQ ID NO:2.

8. (canceled)

9. A fusion polypeptide comprising:
(a) the polypeptide of claim 1; and
(b) a targeting moiety that targets the polypeptide of (a) to a site of complement activation.

10-14. (canceled)

15. A nucleic acid encoding the polypeptide of claim 1.

16. The nucleic acid of claim 15, wherein the nucleic acid comprises the nucleotide sequence depicted in SEQ ID NO:1.

17. A vector comprising the nucleic acid of claim 15.

18. The vector of claim 17, wherein the nucleic acid is operably linked to an expression control sequence.

19. A cell comprising the vector of claim 17.

20-22. (canceled)

23. A method for producing a polypeptide, the method comprising culturing the cell of claim 19 under conditions suitable for expression of the polypeptide or fusion polypeptide.

24. (canceled)

25. (canceled)

26. A pharmaceutical composition comprising:
the polypeptide of claim 1; and
a pharmaceutically acceptable carrier.

27-31. (canceled)

32. A method for inhibiting formation of terminal complement in a biological sample, the method comprising contacting a biological sample with a therapeutic agent in an amount effective to inhibit terminal complement in the biological sample, wherein the biological sample is capable of terminal complement production in the absence of the therapeutic agent and wherein the therapeutic agent is the polypeptide of claim 1.

33. (canceled)

34. (canceled)

35. A method for treating a subject having a complement-associated disorder, the method comprising administering to the subject having a complement-associated disorder a therapeutic agent in an amount effective to treat the complement-associated disorder, wherein the therapeutic agent is the polypeptide of claim 1.

36-41. (canceled)

42. The method of claim 35, wherein the complement-associated disorder is selected from the group consisting of rheumatoid arthritis, a pulmonary condition, ischemia-reperfusion injury, atypical hemolytic uremic syndrome, thrombotic thrombocytopenic purpura, paroxysmal nocturnal hemoglobinuria, dense deposit disease, age-related macular degeneration, spontaneous fetal loss, Pauci-immune vasculitis, epidermolysis bullosa, recurrent fetal loss, multiple sclerosis, traumatic brain injury, myasthenia gravis, cold agglutinin disease, dermatomyositis, Degos' disease, Graves' disease, Hashimoto's thyroiditis, type I diabetes, psoriasis, pemphigus, autoimmune hemolytic anemia, idiopathic thrombocytopenic purpura, Goodpasture syndrome, multifocal motor neuropathy, neuromyelitis optica, antiphospholipid syndrome, and catastrophic antiphospholipid syndrome.

43. The method of claim 42, wherein the pulmonary condition is selected from the group consisting of chronic obstructive pulmonary disorder (COPD), asthma, pulmonary fibrosis, bronchitis, emphysema, bronchiolitis obliterans, and sarcoidosis.

44-46. (canceled)

47. The method of claim 35, further comprising administering one or more additional therapeutic agents for treating a complement-associated disorder.

48. (canceled)

49. A conjugate comprising: (i) the polypeptide of claim 1; and (ii) a heterologous moiety conjugated to the polypeptide.

50-53. (canceled)

54. A kit for use in treating a subject having, suspected of having, or at risk for developing a complement-associated disorder, the kit comprising:
(i) a therapeutic agent selected from the group consisting of the polypeptides of claim 1; and
(ii) a means for delivering the therapeutic agent.

55-63. (canceled)
